(19) United States
(12) Patent Application Publication
(10) Pub. No.: US 2023/0211040 A1
(43) Pub. Date: Jul. 6, 2023

(54) MICROWAVE HEATING APPLIED TO ANIMAL-BASED PRODUCTS

(71) Applicant: A.L.M. Holding Co., Onalaska, WI (US)

(72) Inventors: Drew J. Frederixon, Rockland, WI (US); Gerald H. Reinke, La Crosse, WI (US); Jacob G. Hehir, Holmen, WI (US); Michael R. Byrnes, Sarona, WI (US); Vernon R. Hegg, Plymouth, MN (US)

(21) Appl. No.: 18/093,396

(22) Filed: Jan. 5, 2023

Related U.S. Application Data
(60) Provisional application No. 63/296,946, filed on Jan. 6, 2022.

(51) Int. Cl. A61L 11/00; H05B 6/78; H05B 6/76; C05F 17/80; C05F 17/30; C05F 3/06; C05F 1/00; C05F 1/02; C05F 17/936; B09B 3/50; A23L 3/01

(52) U.S. Cl. CPC A61L 11/00; H05B 6/78; H05B 6/76; C05F 17/80; C05F 17/30; C05F 3/06; C05F 1/005; C05F 1/02; C05F 17/936; B09B 3/50; A23L 3/01

(57) ABSTRACT

A system for processing animal-based material is disclosed that includes at least one microwave generator, at least one microwave guide operatively connecting the at least one microwave generator to at least a first conveyor unit. The first conveyor unit is provided in a first housing that includes at least one opening configured to receive microwave energy via a first microwave guide and the first conveyor unit is configured to receive and process a quantity of animal-based material, which includes heating the animal-based material to a first temperature by applying microwave energy to the animal-based material within the first housing.

### CROSS REFERENCE TO RELATED APPLICATIONS

This application claims the benefit of and priority to U.S. Provisional Patent Application No. 63/296,946, filed Jan. 6, 2022, the entire contents of which is incorporated herein by reference in its entirety.

### BACKGROUND

Microwave energy can be radiated within an enclosure to process materials. Molecular agitation within the material resulting from its exposure to microwave energy provides energy to heat or dry the material. Heating the material using microwave energy can take a certain amount of time based on the quantity, chemical composition of material, moisture content, a desired final heating temperature, moisture content (or other specification), and other factors specific to the intended use of the material in its finally processed form.

Animals are plentiful throughout the earth and provide humans in particular with numerous benefits and uses, including nutrition, fertilizer, among others. In various situations, animal-based materials can be heated in order to provide various processing or other uses of the animal-based materials or products resulting therefrom.

Heating or otherwise applying energy to animal-based materials can be accomplished using microwave energy, which can take a certain amount of time based on the quantity, chemical composition of material, moisture content, a desired final heating temperature, and other factors specific to the intended use of the material in its final, or sometimes intermediate, processed form.

There also exist challenges related to mobile deployment of heating systems for animal-based materials and related material processing, particularly in areas where a reliable permanent power source may not be present.

Some government agencies allocate frequency bands centered at 915 MHz and 2450 MHz for use in microwave heating systems. The intensity of the microwave energy that is permitted to leak is sometimes restricted to less than 10 milliwatts (mW) per centimeter squared.

Many industrial microwave heating applications require that there be access apertures into the enclosure so that materials may be continuously transported utilizing such as, for example, a conveyor unit or other mechanism. There is a desire for suppression of microwave energy from these apertures. Continuous microwave heating arrangements have presented a problem that is more complex than the suppression of microwave energy from a simpler batch microwave system.

While applying microwave heating to materials, such as moisture-containing particles, a problem can include preventing microwaves from escaping to an inlet and/or an outlet/discharge region from a channel or region where the microwaves are applied. This can be handled at present by introducing material through a metal grate including two by two inch (5.1 by 5.1 cm) square channels. The same type of grate and channels can be employed on an outlet end. However, these grates have limitations. For example, granular materials or particles (such as moisture-laden granular materials) are sometimes introduced through a square channel system. In these systems, a blockage or slowdown in the process can occur. For instance, larger chunks of material may have difficulty passing through the grates unless the size of the grate's square metal channels are increased accordingly. A blockage or slowdown in the process can occur. In some cases, animal-based materials, such as animal waste or sewage, can be heated to a certain temperature and/or for a certain amount of time for processing, including for example, treatment. After being treated, such animal waste can be reused for various purposes or disposed of.

Other technological approaches are currently used to prevent potential harmful effects of microwave emissions, but can be less flexible than desirable. For example, other ways of suppressing microwave energy from escaping from a microwave system as a product or material is moving through can include, for example, water jackets or reflectors.

There remains a desire to improve microwave suppression, especially in continuous microwave heating systems. There also remains a desire to provide modular and/or portable animal-based material heating, processing, and treatment systems that can be flexibly deployed as needed, and that can heat materials to a desired temperature, temperature for a time period, a time period, a final moisture content, a reaction point, and/or other target specification or property.

### SUMMARY

This disclosure relates to a continuous microwave-based heating system for improving material processing, especially as applied to various animal-based materials and processing operations thereof. In particular, this disclosure relates to a continuous system for using a microwave heating process at the point of sourcing animals (e.g., livestock), or animal-based materials or precursors to animal-based products, such as a slaughterhouse, farm, veterinarian, zoo, nature reserve, etc. Alternatively, the microwave heating and treatment process can be conducted at a processing facility located a distance from an animal-based material storage or origin site, for example. The disclosed continuous systems can be used in any suitable location, and can be stationary/permanent or mobile in various embodiments. Also disclosed and contemplated are batch-type systems for thermally and/or mechanically treating or processing various animal-based materials from which desirable downstream animal-based products can be produced, including improved consistency, and logistics, among other benefits.

According to the present disclosure, modular heating systems can be configured to include sequentially arranged, multiple conveyor units, mixers, and/or lifting units. Further arrangements provide at least partially parallel arrangements of multiple conveyor units, optionally in combination with sequential arrangements. Disclosed embodiments are fully scalable according to particular desired animal-based material heating, processing, and treatment requirements and specifications, such as of the U.S. Environmental Protection Agency (EPA), (USDA), or other regulatory agencies of various U.S. state, county, city, or municipal governments.

Also disclosed are embodiments of a microwave energy suppression tunnel with one or more flexible or bendable (e.g., steel) microwave reflecting components, such as mesh flaps, for substantially reducing or preventing the leakage of microwave energy from a microwave vessel, e.g., of a conveyor unit, while having a continuous flow of animal-based material through the vessel and suppression tunnels. The suppression tunnels can be installed on the inlet and the outlet side of the vessel and are sized to suppress leakage of the microwaves produced by the microwave system, whatever the size of the animal-based material.

Stated differently, embodiments of the invention include the addition of at least one microwave energy suppression tunnel configured for substantially preventing the leakage of microwave energy from one or more access openings in a microwave energized system while the animal-based material to be heated is flowing continuously through the microwave vessel, including, for example, a trough of a conveyor unit also fitted with a helical auger. The suppression tunnel can be used at material inlets and/or outlets of the microwave energy system, and in some embodiments each suppression tunnel comprises a rectangular, U-shaped, or other suitably shaped tunnel about three feet or more in length installed flat or at an angle of preferably no more than about 45 degrees with multiple plies or layers of steel or other microwave material, such as metallic shielding mesh attached to the inner top of the rectangular or U-shaped tunnel or trough. The size of animal-based materials to be heated can be used as a guideline for adjusting tunnel or trough size for various embodiments. The tunnel and trough of the heating system can be sized and shaped differently in various embodiments.

Flexible or bendable mesh shielding (e.g., in the form of flaps) can be spaced at various intervals and be the same cross-sectional size as the tunnel in which they are mounted. The shielding mesh preferably operates to absorb, deflect, or block various frequency ranges, preferably from about 1 MHz to 50 GHz in radio frequency (RF) and low frequency (LF) electric fields.

Mechanical processing, including comminution (crushing or grinding), milling, sizing, sorting, screening, blending, mixing, cooling/freezing, and/or steps including the introduction of liquids or additives or other materials are also contemplated in order to improve animal-based material processing performance.

According to a first embodiment of the present disclosure, a system for processing animal-based material is disclosed. According to the first embodiment, the system includes a material inlet and a material outlet. The system also includes at least a first conveyor unit associated with at least one of the material inlet and the material outlet. The system also includes at least one microwave generator. The system also includes at least a first microwave guide operatively connecting the at least one microwave generator to at least the first conveyor unit. According to the first embodiment, the first conveyor unit is provided in a first housing that includes at least one microwave opening configured to receive microwave energy via at least the first microwave guide. The system also includes at least one microwave suppression system associated with the first conveyor unit. Each microwave suppression system includes a tunnel associated with at least one of the material inlet and the material outlet, and at least one flexible and/or movable microwave reflecting component included within the tunnel. Still according to the first embodiment, at least a portion of the at least one microwave reflecting component is configured to be deflected as a quantity of animal-based material passes through the tunnel and then to return to a resting, closed position when the animal-based material is no longer passing through the tunnel, and where the first conveyor unit is configured to receive and process the animal-based material, the processing including heating the animal-based material to at least a first temperature by applying microwave energy to the animal-based material within the first housing.

According to a second embodiment of the present disclosure, an apparatus for processing animal-based material is disclosed. According to the second embodiment, the apparatus includes a material inlet and a material outlet. The apparatus also includes a conveyor unit including an auger having an auger shaft provided along an auger rotational axis, the auger configured to rotate in a direction such that a quantity of animal-based material received at the conveyor unit is caused to be transported according to the auger rotational axis. The apparatus also includes at least one microwave energy generator, each microwave energy generator being operatively connected to at least a respective microwave guide configured to cause microwaves emitted by the microwave energy generator to heat the animal-based material within the conveyor unit by converting the microwaves to heat when absorbed by at least a portion of the animal-based material within the conveyor unit. The apparatus also includes at least a first microwave suppression system including a tunnel associated with at least one of the material inlet and material outlet, where the first microwave suppression system includes at least one flexible and/or movable microwave reflecting component within the tunnel, where the at least one microwave reflecting component is configured to absorb, deflect, or block microwave energy, and where the at least one microwave reflecting component is configured to be deflected as the animal-based material passes through the tunnel and then to return to a resting, closed position when the animal-based material is no longer passing through the tunnel. Still according to the second embodiment, the animal-based material is heated using microwave energy, and where the animal-based material is caused to be heated to a target specification by the microwaves emitted by the at least one microwave generator.

According to a third embodiment of the present disclosure, a method of processing animal-based material using microwave energy is disclosed. According to the third embodiment, the method includes receiving a quantity of animal-based material at a conveyor unit, where the animal-based material passes through an inlet microwave suppression tunnel before entering the conveyor unit, where the inlet microwave suppression tunnel includes at least one flexible and/or movable inlet microwave reflecting component within the inlet microwave suppression tunnel, and where the at least one inlet microwave reflecting component is configured to absorb, deflect, or block microwave energy. The method also includes deflecting the at least one inlet microwave reflecting component as the animal-based material passes through the inlet microwave suppression tunnel and then optionally returning the at least one inlet microwave reflecting component to a resting, closing position when the animal-based material is no longer passing through the inlet microwave suppression tunnel. The method also includes transporting the animal-based material using at least the conveyor unit. The method also includes heating the animal-based material within at least the conveyor unit using at least one microwave generator operatively connected to a respective microwave guide configured to cause microwaves emitted by the microwave energy generator to heat the animal-based material within at least the conveyor unit by converting the microwaves to heat when absorbed by at least a portion of the animal-based material within at least the conveyor unit. Still according to the third embodiment, the method also includes causing the animal-based material to exit through an outlet microwave suppression tunnel after the animal-based material is heated such that the animal-based material: a) reaches a first temperature, b) undergoes a reaction, and/or c) reaches a target specification within at least the conveyor unit.

### DESCRIPTION OF THE DRAWINGS

FIG. 1 is a side view of a portable, continuous material processing system, according to various embodiments.

FIG. 2 is a side view of trough and suppression tunnel components of the continuous material processing system of FIG. 1

FIG. 3 is a top view of the continuous material processing system of FIG. 1.

FIG. 4 is a perspective exploded view of the trough of the continuous material processing system of FIG. 1.

FIG. 5 is a top view of the trough of the continuous material processing system of FIG. 1.

FIG. 6 is a top view of an auger for use with the trough of the continuous material processing system of FIG. 1.

FIG. 7 is a perspective view of an alternative trough for use with the continuous material processing system of FIG. 1.

FIG. 8 is a partial cut-away view of the alternative trough of FIG. 7.

FIG. 9 is a top view of the alternative trough of the continuous material processing system of FIG. 1.

FIG. 10 is a perspective view of a multi-conveyor continuous material processing system, according to various embodiments.

FIG. 11 is a top view of the multi-conveyor continuous material processing system of FIG. 10.

FIG. 12 is a perspective view of a mechanical processing apparatus for use with the multi-conveyor continuous material processing system of FIG. 10.

FIG. 13 is a partial cut-away view of the mechanical processing apparatus of FIG. 12.

FIG. 14 is a perspective view of a mobile multi-conveyor continuous material processing system, according to various embodiments.

FIG. 15 is a perspective view of an alternative mobile multi-conveyor continuous material processing system, according to various embodiments.

FIG. 16 is a perspective view of a microwave suppression tunnel, according to various embodiments.

FIG. 17 is a partial cut-away view of the microwave suppression tunnel of FIG. 16.

FIG. 18 is cross-sectional side view of the microwave suppression tunnel of FIG. 16, showing multiple flaps in a closed position.

FIG. 19 is cross-sectional side view of the microwave suppression tunnel of FIG. 16, showing multiple flaps in an open position as flowing material passes the flaps.

FIG. 20 is a front view of an alternative arrangement mesh strip flap for use in a microwave suppression tunnel.

FIG. 21 is a perspective view of the alternative arrangement mesh strip flap of FIG. 20.

FIG. 22 is a cross-sectional side view of a U-shaped microwave suppression tunnel of an outlet side.

FIG. 23 is a cross-sectional top view of the U-shaped microwave suppression tunnel of FIG. 22.

FIG. 24 is a cross-sectional side view of a U-shaped microwave suppression tunnel of an inlet side.

FIG. 25 is a cross-sectional side view of a rectangular microwave suppression tunnel of an inlet side.

FIG. 26 is a cross-sectional top view of a rectangular microwave suppression tunnel of FIG. 25.

FIG. 27 is a cross-sectional side view of a rectangular microwave suppression tunnel of an outlet side.

FIG. 28 is a schematic side view of a hardware detail section of a non-looped microwave absorbing flap with a mesh attached to a microwave suppression tunnel.

FIG. 29A is a cross-sectional end view of a U-shaped microwave suppression tunnel configuration with a top-mounted pivoting mesh flap in a closed position.

FIG. 29B is a cross-sectional end view of the U-shaped microwave suppression tunnel configuration of FIG. 29A with the mesh flap in a partially open position.

FIG. 29C is a cross-sectional end view of the U-shaped microwave suppression tunnel configuration of FIG. 29A with the mesh flap in a fully open position.

FIG. 30A is a cross-sectional end view of a rectangular microwave suppression tunnel configuration with a top-mounted pivoting mesh flap in a closed position.

FIG. 30B is a cross-sectional end view of the rectangular microwave suppression tunnel configuration of FIG. 30A with the mesh flap in a partially open position.

FIG. 30C is a cross-sectional end view of the rectangular microwave suppression tunnel configuration of FIG. 30A with the mesh flap in a fully open position.

FIG. 31 shows various alternative chute cross-sectional shapes of a microwave suppression tunnel.

FIG. 32 is a flowchart of a process according to various embodiments of the present disclosure.

FIG. 33 is a detail view of an RFI shielding mesh according to various embodiments.

FIG. 34 is another view of the RFI shielding mesh of FIG. 33.

FIG. 35 is a transmission damping chart of the shielding mesh according to FIG. 33.

FIG. 36 is a detail view of another shielding mesh according to various embodiments.

FIG. 37 is another view of the shielding mesh of FIG. 36.

FIG. 38 is a transmission damping chart of the shielding mesh of FIG. 36.

FIG. 39 is a perspective view of another embodiment of a portable, continuous material processing system.

### DETAILED DESCRIPTION

According to the present disclosure, many challenges currently exist related to processing and logistics of animals, animal materials, animal-based materials, animal by-product materials, animal-derived materials, and related materials. A broad definition of animal is contemplated herein, including but not limited to: mammals, fish, reptiles, amphibians, birds, and invertebrates. Animals as contemplated herein include all species in the biological kingdom animalia. For clarity and as used herein, an animal-based material generally refers to a received input to be processed or being processed as disclosed herein, and an animal product or animal-derived product is an output product made, e.g., based on at least some processing of the animal-based material as an input. In various embodiments, an input animal-based material input can also be referred to as the animal-based material in output, processed form. Products that are derived from or related to animals are sometimes referred to as products of animal origin (POAO), which are contemplated herein.

Examples of animal-based (or related) products include any products or output materials that are made based on animal-based or animal-related material derived from or including the body, body parts, waste originating from animals, or other related materials that are related to, directly sourced from, or produced by one or more individual or type of animal. Some non-limiting examples of animal-based or animal-related materials are animal fat, flesh, meat, blood, feathers, bone, tendon, hair, skin, organs (e.g., liver, lung, kidney, brains, spleen, tripe, intestines, heart, etc.), milk, eggs, larvae, grease, oils, isinglass, rennet, urine, fecal matter, secretions, etc.

Animal-based materials contemplated herein include any animal by-product materials, including materials and products based in part or wholly on one or more type of and/or individual animals. Various animal by-product materials contemplated herein include animal-based materials and animal-based products resulting from processed or treated animal-based materials, such products in some cases being intended for human consumption, usage, or the like.

Some animal by-product materials contemplated include any as defined by the United States Department of Agriculture (USDA), which includes materials harvested or products manufactured from livestock (typically other than muscle meat). Muscle meat as used herein is also contemplated as a type of animal-based material under a broader definition. Similarly, in the European Union (EU), animal by-products (ABPs) can be defined as materials from animals that humans do not typically consume. Animal by-products (materials) also include animal carcasses and parts thereof, which can be received from slaughterhouses (such as slaughterhouse waste), or any other source of animals or carcasses thereof. Animal waste, including dry and liquid manure therefrom, is also considered an animal-based product, herein. One example of animal waste is recycled manure solids (RMS). Another is dried manure solids (DMS). In optional embodiments, the terminology of animal-based materials can broadly include fossilized or decomposed animals, such as petroleum products, or any crop grown in soil that is fertilized by animal remains or manure.

In various embodiments herein, where an animal-based material contains animal manure, the manure can be converted into an output product resulting from various thermal processes. Examples of thermal and/or thermochemical processes herein include pyrolysis, gasification, combustion, and the like. Outputs can include liquid, gaseous, and/or solid products, and the output products can be used as fuel (e.g., dry dung fuel), etc. The USDA

Animal Product Manual of the Animal and Plant Health Inspection Service is hereby incorporated by reference for all purposes herein, and any examples therein derived from or related to animals are to be understood to be animal-based materials, as inputs, outputs, specifications, best practices, processing details and options, etc. as used herein.

Animal-based materials can be used to produce output products such as food for human or animal consumption. Food safety and sanitary production are important aspects of animal-based material processing. Avoiding and eliminating bacteria and providing a sanitary food product is desirable and, in many cases, necessary. Therefore, various animal-based materials can be processed and/or treated according to various embodiments herein, including heating materials to a target temperature, a target temperature for a target time, to a reaction point, or other threshold heating point or energy application goal. In alternative embodiments, a target time of processing can be a goal or specification to be achieved in processing, with less or no regard for actual power levels, energy delivered, or final temperatures reached.

Animal-based materials to be heated using microwave heat as discussed herein includes viscous or non-viscous received animal-based materials. For example, heating viscous materials such as ground animal meat in a vessel has certain aspect that can be different than heating non-viscous, homogeneous liquids of animal-based materials. Heat transfer characteristics to various materials vary based on material properties, such as stickiness or various surfaces during heating. Various animal-based materials contemplated herein include various slurries received as such and/or produced therefrom.

Animal bedding, such as cow bedding, can be produced from animal-based materials. In fact, cow cubicles bedded with, e.g., manure separates, can be preferable to bedding formed from straw, sawdust, sand, wood chips, etc. For example, various bedding materials can be produced, e.g., locally, by physically separating recycled manure solids (RMS) or other animal-based materials. Cow bedding is commonly used for cows, especially dairy cows, in relatively dry climates. Improved cow comfort can result from cow bedding and the quality thereof. Lower prevalence of organisms in reused cow bedding can have advantages, and therefore thermal processing of animal-based materials to produce low pathogen and sanitary cow bedding as an output product is desirable. Improved cow udder health can result from high-quality cow bedding, including bedding made from recycled manure. In European Union Animal By-Products Regulations (Regulation 1069/2009) defines an example standard by which animal manure can be reused as a technical product, such as animal bedding. According to this regulation, a safe end use of a product derived from animal by-products is permitted under conditions which pose no unacceptable risks to public and animal health.

According to the present disclosure, such standards can be met and/or exceeded using microwave thermal processing. Additional details related to recycling manure, especially solids, for reuse as cow bedding is found in the article titled “Recycling manure as cow bedding: Potential benefits and risks for UK dairy farms.” Leach, K. A., Archer, S. C., Breen, J. E., Green, M. J., Ohnstad, I. C., Tuer, S., & Bradley, A. J. (2015). Recycling manure as cow bedding: Potential benefits and risks for UK dairy farms. Veterinary journal (London, England: 1997), 206(2), 123-130. https://doi.org/10.1016/j.tvjl.2015.08.013, and “Bedding Options for Dairy Cows,” UMass Extension Crops, Dairy, Livestock, Equine, CDLE Pub. 11-48, which are hereby incorporated by reference in their entireties for all purposes.

Also contemplated and incorporated herein in its entirety is Title 9, Chapter III, Subchapter E, Part 431 of the US Code of Federal Regulations, entitled: “Thermally Processed, Commercially Sterile Products,” any definitions, regulations, specifications, or details therein being applicable to the systems and methods herein as applied to heating of animal-based materials.

Bedding can be recovered from manure. Manure can be scraped, flushed, or otherwise extracted from animal stalls or cubicles. The manure, an example of animal-based material, is then subject to anaerobic digestion to reduce the bacteria content and odor of the material. A slurry is then formed from the manure, which can then be subject to one or more solid/liquid separation processing step. For example, a screw press, centrifuge, or slope screen can be used to separate coarse manure fibers from a liquid portion. Microwave heating using disclosed methods and systems can occur at this step. The resulting animal-based waste material is a recycled manure solid that can be used as bedding. A further drying and/or processing step using microwave heat can occur to more completely dry the manure material before use as bedding. Preferably, bacterial content of the material is minimized, e.g., by microwave heating. Pasteurization of the animal-based material can also be performed. The article entitled “Bedding Recovery From Manure: The Solution To Livestock Bedding” by Carlson, Carrie and Reckinger, Nick; FEECO INTERNATIONAL, URL: https://feeco.com/bedding-recovery-from-manure-the-solution-to-livestock-bedding/(Accessed Dec. 28, 2021) is also hereby incorporated by reference for all purposes herein.

Further contemplated uses of animal-based materials, such as manure, are listed by the US Environmental Protection Agency (EPA). According to the EPA, various components of manure that can be reused include nutrients, organic matter, solids, energy, and fiber. Also, according to the EPA, various beneficial uses of manure, which are also contemplated herein, include: compost, fertilizer, biomass (through conversion, such biomass being usable as animal feed, soil amendments, or fertilizer), soil amendments/structuring, bedding, biogas, bio-oil, syngas, peat substitute, paper, and building materials. Additional detail from the EPA can be found at URL: https://www.epa.gov/npdes/animal-feeding-operations-uses-manure, the contents of which are hereby incorporated by reference for all purposes.

Animal waste material is an example of animal-based material to be heated and/or processed as described herein. Animal-based waste can include any liquid, solids, or slurries thereof, can be heated and/or processed using microwaves as described herein. Animal waste material as used herein can include waste produced by animals, such as manure and/or urine produced thereby, but can additionally or alternatively denote animal various parts of the animal itself or materials or products derived therefrom, e.g., during butchering or processing of animals, animal parts, meat, entrails, etc.

In various embodiments, this disclosure relates to methods and systems for processing and/or heating animal-based material, including waste material using microwaves. Animal waste material as used herein includes animal-related biowaste, biosludge, and other waste such as fecal matter, and waste activated sludge obtainable or obtained from aqueous animal waste streams, among other types of waste material. Also contemplated herein is the production of various products prepared from animal waste material, such as solid protein feed products, and methods of preparing such products. Disclosed methods are useful in that they can enhance animal waste and water remediation and provide for a raw material that may be used for the production of various products, including for example an animal feed, pet food, a human food product, animal bedding, fertilizer, etc. Therefore, animal-based material can be treated for reuse or safe disposal in some form following treatment.

In some disclosed methods for processing animal-based materials, including waste materials, the material can be obtainable from wastewater or livestock processing plants. These waste materials are useful in that they can provide output products or novel waste activated sludge preparations that are substantially free of live microbial organisms and optionally contain a high content of digestible protein. In other embodiments, an end product can be combustible, edible to humans or animals, fertilizer, bedding, such as livestock (e.g., cow/bovine/cattle or the like) bedding, etc.

An example material processing facility can benefit from a way to kill pathogens in received animal-based waste (e.g., biowaste). Received waste can include animal-based waste as defined broadly, e.g., animal body parts (e.g., meat, organs, etc.) and/or urine/fecal waste, or any suitable type of animal-based waste material containing at least some water or moisture, preferably flowable. The animal-based waste material can be a waste sludge received from any animal-related residential, commercial, or industrial site or process. The animal waste material can contain any suitable liquid, flowable, or semi-liquid waste product. Examples of the animal waste can be carried and flowed with water, and can include at least some animal manure in sludge form. Once the pathogens in the biowaste are neutralized from heating the biowaste can be repurposed, e.g., sold for land application, farming, among other uses.

Disclosed microwave waste heating systems such as described herein is an ideal candidate for treatment of the received animal biowaste, such as to create an output product that meets specific requirements for land application.

Also contemplated herein as examples of animal-based materials are so-called “rendered” animal materials, either to be received prior to microwave-based heating or following such heating. Rendering is known in the art as a process in which waste animal tissue is converted into stable, usable materials. Rendering can be performed on restaurant grease, butcher shop trimmings, expired meat from grocery stores, and so forth. Common animal sources for rendering include beef, pork, mutton, and poultry. Heating various animal-based materials can also cause a caramelization and/or coagulation in various embodiments.

According to the present disclosure, a problem currently exists in the art relating to treating and processing animal-based materials by heating the animal-based material (or related or derived composition) to a desired temperature using microwave energy while continuously moving the animal material during heating. For example, an animal-based material to be heated to varying degrees, such as heating to a point such that all, substantially all, or a substantial percentage of pathogens within received animal-based materials are exterminated by heating to a certain temperature for a certain amount of time. In other cases, lower levels of heating are used, such as pasteurization and other lower-heat thermal processing.

Certain contemplated configurations use a “batch” style heating and processing system. In batch systems, a quantity of animal-based material is heated and/or mixed together as a single stage and then is dispensed. It is often desirable to have more flexibility than a batch-style heating system affords because flexible operation of the heating and/or mixing system is preferred. Therefore, continuous type heating and/or mixing systems can be preferable because they can provide greater efficiency, control, and flexible scalability and operation, among other benefits. Also disclosed and contemplated are batch-type systems for heating animal material.

Other challenges also exist in the art relating to microwave emissions escaping a heating system. In a continuous production system, microwave energy leakage can be particularly undesirable and challenging.

Another common complication in the art relates to rapid distribution and deployment of heating apparatuses to remote or non-grid-connected regions or situations. Microwave-based heating is generally more portable than other types of heating apparatuses and allows for portable generator use to power the microwave heating units (e.g., microwave generators) and system if mains or grid power is not readily accessible. Some examples of situations where grid power is not available include rural or remote areas, or other areas that have temporarily lost a grid power connection.

According to the present disclosure, portable, modular, parallel, and/or sequential heating and/or processing conveyor units can provide a modular, scalable, and portable system for heating an animal-based material even in remote, or otherwise off-grid locations. Sharing of animal-based material processing systems between multiple locations and/or facilities is also contemplated. A portable system can require little or no assembly to reach operability once transported to a site for treating or processing animal-based materials. Stationary, semi-permanent, and permanent embodiments are also contemplated. Primarily stationary systems can nevertheless be transported, e.g., in components or parts, to various locations for final assembly.

Various mixers and/or lifting conveyors can be used in-line with the conveyor units as suitable. Packaging various operative components within or attached to containers or other housings, such as shipping containers, can further simplify and streamline rapid and simple distribution, setup, and operation for portability when utilized. Portable animal-based material processing systems disclosed herein can be integrated, attached, or otherwise associated with any of various trailers, trucks, machinery, trains, and the like.

Also, according to the present disclosure, various microwave suppression systems and features, such as included in or related to inlet/outlet tunnels can be sized to accommodate the size of the flow or passage of whatever animal-based material is being heated and/or processed, such as various types and sources of animal-based materials and the like. In some cases, a microwave heating system of the present disclosure can be configured to process/heat about 100 tons of animal-based material per hour or more according to various specifications and standards, although it would be obvious to one skilled in the art that the process could be scaled to accommodate quantities of less than 100 tons of material per hour and reach target specifications. For example, certain types of animal-based material can include a greater amount of moisture than other types of material. A rated capacity of a system can be selected and configured based on an end goal of a particular facility and/or municipality. For instance, one goal may be to kill or otherwise affect pathogens found in animal-based material or to convert one type of material to another using thermal, chemical, and/or mechanical processing. To kill pathogens within the animal-based materials, animal material may be heated to reach about 180° F. (82.2° C.) for approximately 1-10 seconds. These specifications may therefore require less energy and allow for higher throughput than certain other specifications. End throughput and configuration can be determined based on end goals of a user, and whether water content of the material is to be vaporized through boiling.

One or more microwave suppression systems (e.g., tunnels or chutes) including one or more (e.g., flexible and/or movable) fabric and/or mesh flaps can be used at one or more material inlet/outlet openings within a microwave-based heating system in order to reduce microwave emissions that would otherwise reach the outside of the heating system. Each microwave suppression system can include a flap or series of flaps that are capable of and configured to cover one or more inlets and/or exits from a microwave heating system. Flexible or bendable mesh shielding (e.g., in the form of flaps) can be spaced at, for example, about six-inch (15.2 cm) intervals and the flaps be the same cross-sectional size as the tunnel in which they are mounted. The microwave suppression systems can prevent or suppress the escape of microwave emissions from the material heating system. Therefore, one or more of the fabric and/or mesh flaps can be positioned at outlets and/or inlets of the continuous microwave material heating system. Each flap can be generally shaped to conform to a shape of a corresponding suppression tunnel, chute, component thereof, or the like. Outlets and/or inlets of the continuous microwave heating system can include one or more suppression tunnels. In particular, moisture-laden material, animal-based materials, and/or other component particles or material can be allowed to enter into the heating region of microwave heating while microwaves are simultaneously substantially prevented from escaping a heating trough via the suppression tunnels within the system. As multiple modular heating and processing conveyors can be arranged sequentially and/or in parallel, various material inlets and outlets are particularly suitable for microwave suppression systems, including tunnels and other related features. In preferable embodiments, separate suppression systems such as tunnels are supplied and connected to both an inlet and an outlet of a system. In other embodiments, additional suppression tunnels or related features can be included intermediately within a material flow path or otherwise to the system such that more than two such suppression systems are included in order to maximize microwave suppression from any number of openings in the system.

It is known that microwave energy is particularly efficient for heating water (e.g., water molecules), which leads to efficient microwave heating of materials that include at least some of such water molecules. Animal-based materials in some embodiments disclosed herein can contain about 70% water, although embodiments containing less than 70% or more than 70% water are also contemplated herein. Water can escape a material in the gaseous form of steam when the water is heated to its boiling point (e.g., about 212° F. or 100° C.). Steam can escape from a heating system through natural ventilation, and in some cases by forced ventilation, through positive or negative pressure applied to the system (e.g., an air blower or fan to expedite or assist ventilation). Vents can also be added to improve ventilation and facilitate steam escape characteristics. Excessive quantities of water can have a negative effect on heating animal-based materials. Furthermore, heat exchangers can be used to reclaim heat released as steam (or otherwise) during microwave heating processes, and in particular heat that is emitted from the phase change (e.g., boiling) of water when the material containing at least some water is heated.

In some typical cases, animal-based materials can be about 5-90%, or in some cases about 50-80% water content by weight, or any other percentage according to each situation.

Heating a quantity of animal-based material to a temperature above the boiling point of water (about 212° F. or 100° C.) can therefore be less efficient because the water particles boil off and escape as steam. During heating organic or inorganic materials to certain temperatures, e.g., at or above a boiling point of water, the number of small dipole molecules (e.g., water) that the microwaves can easily heat through oscillation can decrease. Heating of the animal-based material then becomes reliant on the microwaves oscillation larger particles which may require more energy. If the animal-based material being heated is for example, animal waste or other water-containing material, more water is removed from the heated waste material as heating temperature increases. A phase change of liquid water to gaseous steam can occur around 180-212° F. (82-100° C.) depending on air pressure or vacuum, and it can be desirable to heat a material, e.g., an animal-based material, to about 180-212° F. (82-100° C.) or even to about 225-275° F. (107-135° C.), according to various embodiments. A target heating temperature can be determined based on various goals or targets according to a particular situation and/or need. In some cases, a target temperature of about 180° F. (82° C.) can be sufficient for elimination of pathogens. Where a goal is overall volume reduction and/or water removal, a target temperature can be about 212° F. (100° C.). Steam that is produced from the heating can escape the heating system via vents once the phase change occurs. According to various embodiments contemplated herein, steam/vapor and/or other heat produced and/or emitted during microwave heating can be captured for re-use using one or more air-air, and air-liquid heat exchangers or the like. The steam can exit the system by natural and/or forced ventilation. In some cases, there may be least waste emissions below about 160° F. (71° C.), or at a maximum below about 270-275° F. (132-135° C.). Waste emissions are dependent on final material temperature and water content and increase with percentage water and temperature. In some embodiments a scrubber system can be implemented that is configured to trap or scrub emitted steam, vapor, particulates, and/or odors that result from animal-based material processing.

In some embodiments, one or more components of an animal-based material processing system can be sealed and/or pressurized, e.g., in a pressurized heating vessel of a microwave material processing system. Pressurization of system components can provide benefits, including containing any steam produced from water content of animal-based material during microwave heating of the material and providing efficiencies by not discharging heated steam and resulting increased pressures. In yet further embodiments, heat conductivity of gaseous steam/water molecules provides increased heating efficiency during material processing described herein. In yet further embodiments, heated steam and/or heated material can be used with heat exchangers in order to transfer thermal energy from a position to another position, or the like.

According to various embodiments the material to be heated and/or processed is an animal-based material, other material, combinations, mixtures, and variations thereof. In certain embodiments the material can be various particles, such as particles to be heated. The material can be composed of various particulate materials and can be flowable, including liquid, semi-solid, or partially or non-flowable without further processing in other embodiments.

The animal-based material(s) can be various primary (non-waste) or waste animal-based materials. If the animal-based material contains animal waste, e.g., animal fecal waste, biowaste, wastewater, by-products, or any other type of animal-based agricultural, natural, commercial, or industrial animal-related waste, this can be thermally processed alone or in combination with various other primary animal-based materials, such as animal body parts and the like.

Materials to be processed herein can have an initial, first maximum or average particle (or clump) size or viscosity. The initial, first particle or clump size or viscosity can be reduced to a second, smaller maximum or average particle or clump size or viscosity by a component or feature of at least one of the first and second conveyor units, such as a baffle as described herein, or any other suitable mechanical or other processing component for reducing particle or clump size or viscosity as known in the art, such as an impactor, shredder, mixer, mesh, mill, brush, or the like. If present, the impactor, shredder, mixer, mesh, mill, lifter, or brush can be separate from the first and second conveyor units. Torque load on a motor in a conveyor unit can be sensed and optionally used as a proxy for viscosity and/or clumping of material being processed. Torque load and power can also be controlled in response to an input from a motor controller or the like.

According to various embodiments, and as discussed above, the received animal-based material to be processed or treated typically contains at least some water. Optionally, the material contains less than ninety percent water by weight. In various further examples, the animal-based material contains at least five percent water by weight. In yet further examples, the animal-based material contains less than ten percent water by weight. In yet further examples, the animal-based material contains between twenty and ninety percent water by weight. In even yet further examples, the animal-based material contains between about fifty and ninety percent water by weight. A target water percentage by weight can be defined by a specification or the like, and in some embodiments as described herein, the methods of systems described herein can be used to output an animal-based product with a desired water content optionally lower than a received animal-based material.

As discussed herein, in at least some embodiments, one heat exchanger apparatus configured to recover a heat byproduct from the animal-based material. In some examples the heat byproduct is recovered from the steam resulting from a heating of the water within the animal-based material.

In some embodiments, one or more additives, substances, or other materials can be added to animal-based material to be heated and at various stages during, before, or after processing. Various additives can provide a number of different qualities when added to material being processed. For example, additives can increase microwave energy absorption and efficiency during heating, can reduce odor or other animal-based emissions, or can chemically alter the material for any reason.

In some embodiments, a continuous microwave heating process can include ramp-up time, hold time, process time (e.g., based on time and temperature of processing), and various heating peaks. Mixing of animal-based materials of differing physical properties can improve performance during microwave heating, according to some embodiments. In other cases, mixing of animal-based materials is done out of convenience and processing heat and/or speed can be controlled based on the received mixture of various animal-based materials.

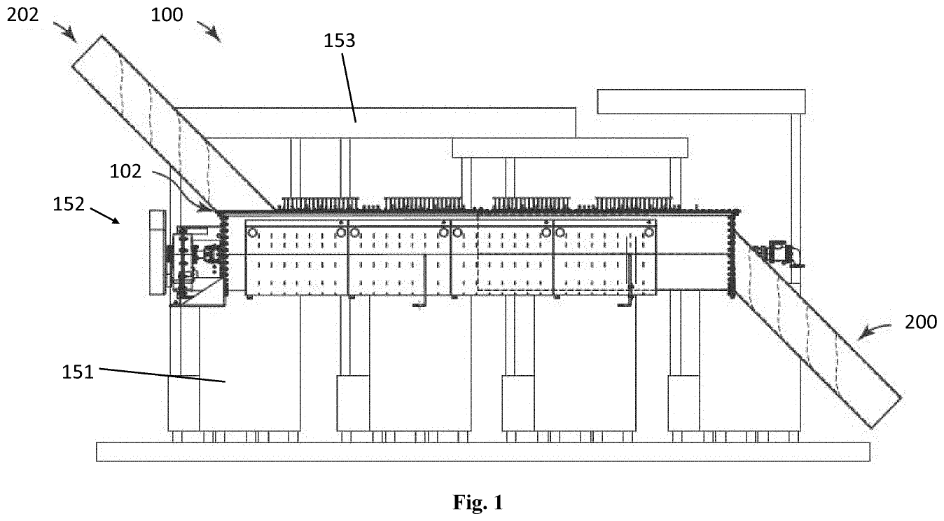

A continuous microwave heating system can be sized in order to get a desired throughput and to accommodate the physical size of the animal-based material being heated. This can be due to limitations, such as with existing heating, mixing, and tunnel designs in view of target treatment specifications as described herein. An example (e.g., aluminum or stainless steel) mesh or fabric flap design of a microwave outlet suppression tunnel 200 as shown in FIG. 1 (and as explained in greater detail below) is better suited for high-volume continuous flow of various sized and consistencies of animal-based materials. Microwave outlet suppression tunnel 200 is an example of a microwave suppression system as used herein. Also as shown in FIG. 1, multiple flaps can be used in a single microwave outlet suppression tunnel 200, e.g., four positioned sequentially as shown. Each flap is preferably shaped to conform to a shape of a corresponding outlet suppression tunnel 200, chute, or the like.

Drying, heating, treating, converting, pasteurizing, sterilizing, transforming, and/or mixing (collectively “processing”) of materials such as animal-based materials is contemplated herein. However, any one type of suitable material can be heated, such as any other animal-based material that can be heated, and conveyed or flowed through a microwave heating system. Animal-based food materials products, which can be defined as a material either before or after having been consumed by a human or animal, can also include certain plant-derived products, animal-derived products, and the like, which can also be heated and dried through the application of microwave energy. Additionally, any known processes, including sanitization, pasteurization, etc. of various animal-based materials is also contemplated. In fact, animal-based materials can be sanitized and heated such that the animal-based material becomes suitable for safe and beneficial re-use. Other applications of the microwave heating of animal-based materials are also contemplated. It may be desirable to substantially sterilize an animal-based material such that it can be adaptively reused as a product to be resold or otherwise used, such as fertilizer, animal feed, human-suitable food, animal bedding, etc. Certain regulations and practical requirements require a certain temperature to be reached and sometimes for a certain amount of time to reach a practical specification, although certain reactions and the like can have varying precise specifications in some cases.

Various embodiments of heating and/or processing systems discussed herein can have various total weight, and/or throughput capacities, depending on dimensions, power capacity, arrangements, and the like. In some embodiments, a continuous material processing system discussed herein has a capacity of about 10-1000 U.S. tons (9.1-907.2 metric tons) of animal-based material per hour. In further embodiments, the capacity can be between 50-100 U.S. tons (45.4-90.7 metric tons) of animal-based material per hour.

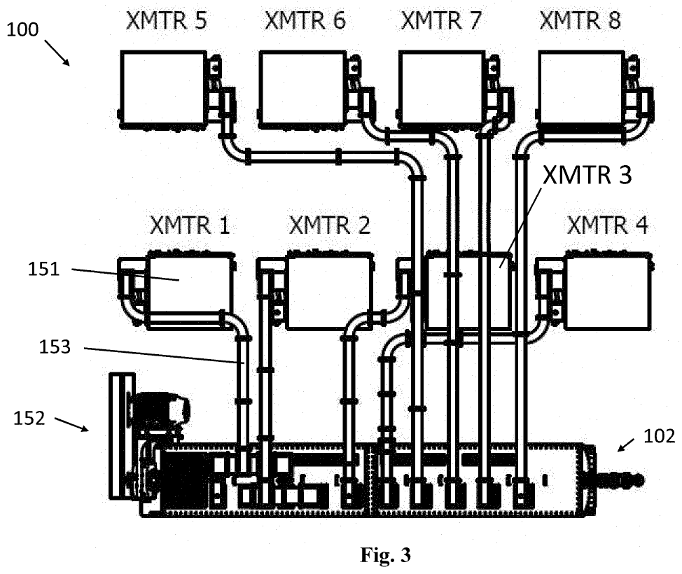

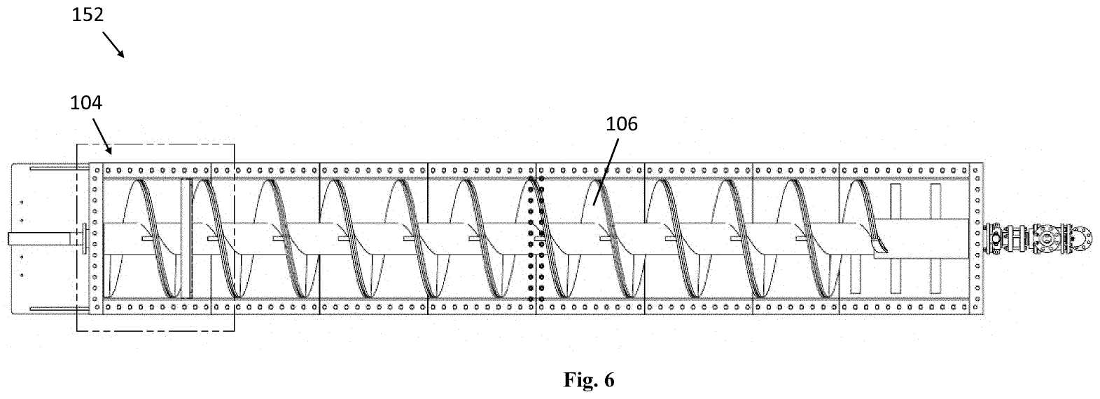

FIGS. 1-9 illustrate an embodiment of a continuous animal-based material processing (or treatment) system 100 having a housing, vessel, or trough 102 (as shown in FIGS. 1-5) (or alternative trough 104 as shown in FIGS. 6-9) including a microwave heated apparatus with one or more microwave heating units 151 each with at least a corresponding waveguide 153 to define a guide path for microwaves (see e.g., FIGS. 1 and 3). In various embodiments, the processing system 100 can be portable. The trough 102 can be made of any of various steel alloys, including stainless steel, and can be either coated or uncoated, or any other suitable substance or combination or alloy of substances. The trough material can be selected to minimize or eliminate reactivity to various animal-based materials and the like. The continuous treatment system 100 also preferably includes at least an outlet suppression tunnel 200, as shown. As shown, the continuous treatment system 100 also includes a housing including a trough 102 including one or more microwave heating units 151, a conveyor-unit-based system such as including an auger 106, an inlet suppression tunnel 202, and the outlet suppression tunnel 200. Examples of these components are described in greater detail herein.

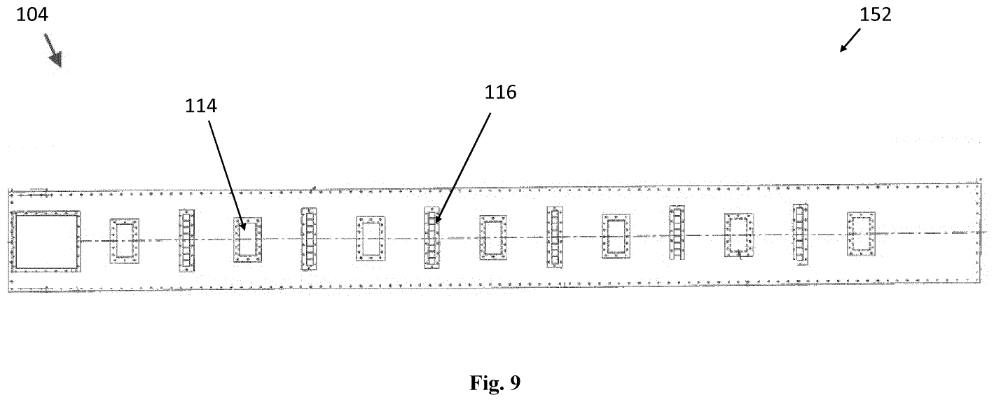

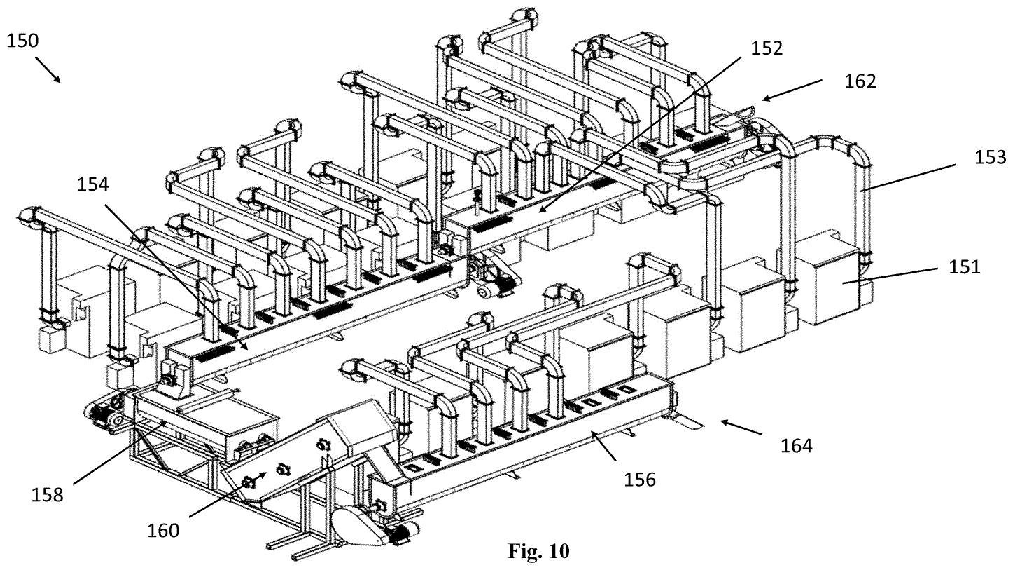

According to FIGS. 1-9 a single conveyor unit continuous heating and/or processing system 100 is shown, although in various embodiments herein (e.g., FIGS. 10, 11, and 14) it is also shown that multiple conveyor units can be assembled sequentially. Conveyor units can therefore be assembled sequentially, but also in parallel, or both in order to achieve a desired throughput for a given conveyor unit size and/or heating capacity; or in order to achieve a desired heating capacity and throughput for a production rate needed to fulfill specification and standards requirements for heating a quantity of animal-based material. Therefore, arrangements and the like can be adjusted for a given conveyor unit specification by introducing multiples of the conveyor unit and/or arrangements thereof. For example, running two conveyor units in parallel can offer twice the heating capacity and/or throughput of processed material compared to a single conveyor unit, provided suitable microwave heating units are provided.

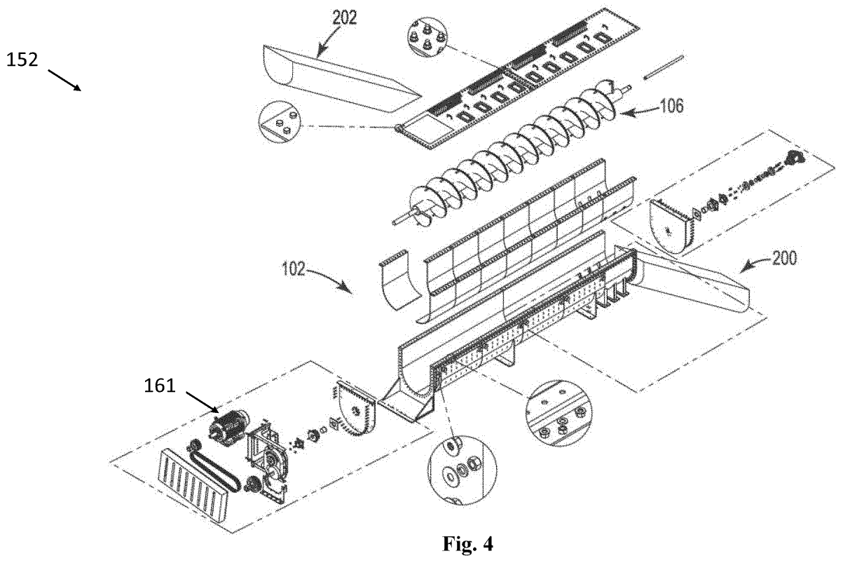

Shown best in FIGS. 4, 6, and 8, a helical auger 106 or (e.g., a helical screw) is one option for a conveyance mechanism by which material particles can be caused to pass through the housing trough 102 longitudinally. The auger 106 can be completely or partially covered in particles (e.g., any other form of animal-based material) to be heated during operation, but the particles are not shown for clarity. The auger 106 can be a heated auger, and in some examples can be a jacketed auger (e.g., where an auger has a hollow fighting that heating fluid is run through as desired). An interface of the auger 106 and trough 102 of the system 100 can be sealed and protected such that any lubrication is substantially isolated from any material being processed, preferable reducing likelihood of the auger 106 jamming or wearing prematurely. In some examples as a smaller auger 106 can be more easily sealed off from exposure to lubricants and the like.

The outlet suppression tunnel 200 can be connected to an outlet and/or inlet of trough 102. The trough 102 can be level or can be canted at an angle to the horizontal plane according to various embodiments. An angled trough 102 (and/or auger 106 in some embodiments) can facilitate movement of the material during processing by utilizing gravity assistance to flow downhill. An example trough 102 can be about twelve feet (3.66 m) long and five feet wide (1.52 m), although any suitable size and/or shape is also contemplated.

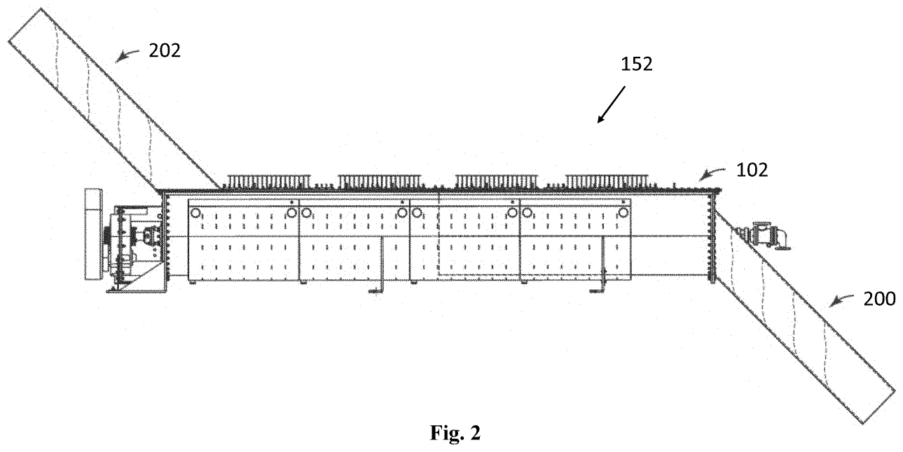

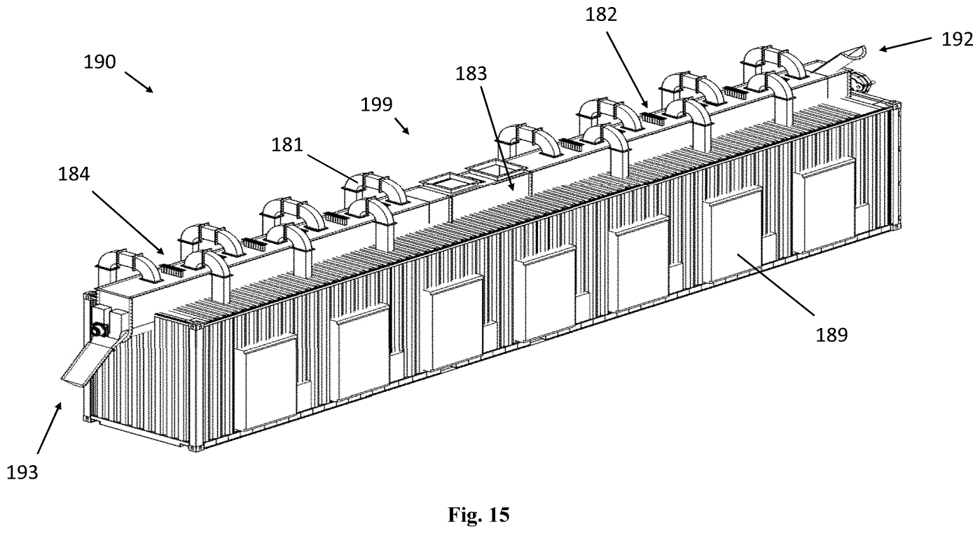

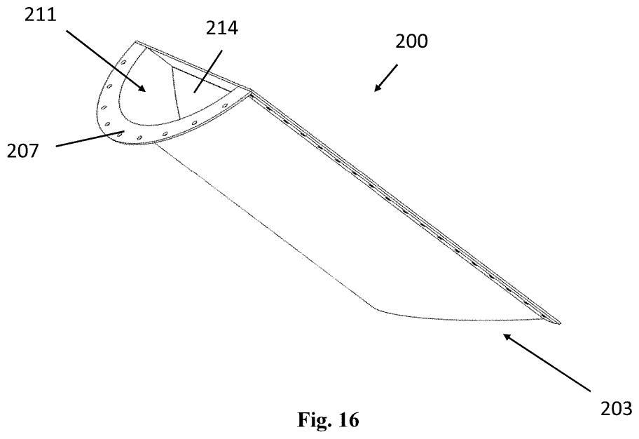

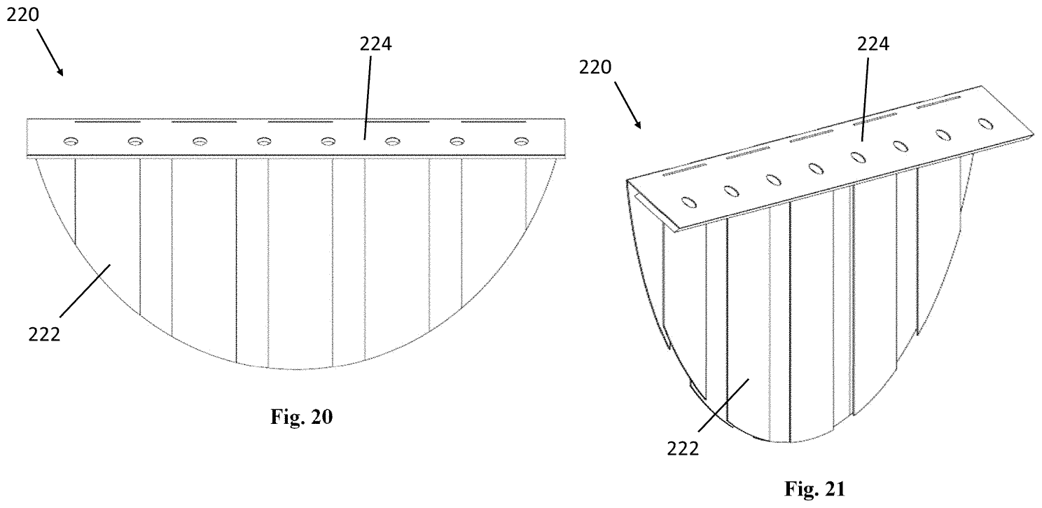

FIGS. 2-9 show various components of the trough 102, auger 106, inlet suppression tunnel 202, outlet suppression tunnel 200, and other components of the system 100 in greater detail. Selected embodiments and variations of the inlet suppression tunnel 202 and the outlet suppression tunnel 200 and components thereof are shown in yet greater detail with respect to FIGS. 16-31. Furthermore, various embodiments of multiple-conveyor microwave-based animal-based material heating systems are shown with reference to FIGS. 10-15.

FIG. 3 shows a general configuration of a single-conveyor unit 152, continuous heating system 100 of the present description, including eight microwave heating units 151, a microwave waveguide 153 for each heating unit 151, an auger-based continuous heating assembly with trough 102, and various other components. In particular, FIG. 3 shows an example including eight microwave heating units 151 labeled as XMTR 1, XMTR 2, XMTR 3, XMTR 4, XMTR 5, XMTR 6, XMTR 7, and XMTR 8. More or fewer microwave heating units 151 (and corresponding waveguides 153) can be used in alternative embodiments. A number of waveguides 153 and therefore microwave generators 151 used with a trough 102 can be limited by a surface area on top (or other side) of the trough 102, including any vents, inlets, and/or outlets included thereon. In some examples 1-30 waveguides 153 can be utilized for each conveyor unit, and in more specific embodiments 7-10 waveguides can be utilized for each conveyor unit.

One example microwave heating unit 151 can be a microwave power system sourced from Thermax Thermatron. The microwave heating units 151 can have a variety of shapes and sizes according to the requirements of the continuous heating process and system 100. Each microwave heating unit can apply about 100 kW of power to the material being heated and preferably operates at about 915 MHz. In various examples, various quantities of microwave energy can be received by the material while in a conveyor unit.

Various conveyor units described herein (e.g., conveyor unit 152) can have a nominal weight capacity of about 500-40,000 lbs (500-18,144 kg). In some examples, the conveyor units can each have a weight capacity of about 8,500 lbs (3,856 kg) of material at a point in time.

Various example waveguide 153 configurations and embodiments for a single conveyor unit 152 are shown in FIGS. 1 and 3. The various waveguides 153 can be configured to bend and be routed such that no two waveguides 153 collide, and in some cases the waveguides can be configured to minimize turns or bends in the waveguides, as practical. Similar waveguide 153 configurations can be adapted for use with multiple-conveyor unit animal-based material processing systems described below. Each microwave heating unit 151 can optionally be connected to more than one waveguide 153.

Still referring to FIG. 1, a side view of the continuous heating assembly is shown, including an example inlet suppression tunnel 202, outlet suppression tunnel 200, and trough 102 of system 100. Although not shown, the trough 102 can be generally mounted or positioned, or provided with a shape generally including an angle relative to horizontal to facilitate animal-based material movement or production during heating and/or conveying material for processing described herein, e.g., by at least partially utilizing gravity to move the material through the trough 102. Non-stick coating can be applied to the trough 102, such as to an interior portion of the trough 102 such that animal-based material is less prone to stick and resist movement during processing.

FIG. 4 is an exploded view of system 100. Shown is a conveyor motor 161 for rotating the auger 106, the housing trough 102 for holding and carrying the material to be heated, the inlet suppression tunnel 202, the outlet suppression tunnel 200, and various other components. The conveyor motor 161 can be an electric, brushed or brushless, induction or permanent magnet, variable reluctance, etc. motor and can utilize alternating current (AC) or direct current (DC) power of any voltage or power as suitable. Any other suitable type of motor, including an internal combustion engine or gas turbine, can also be implemented. In particular, FIG. 4 provides a more detailed view of system 100, including the trough 102, auger 106, inlet suppression tunnel 202, outlet suppression tunnel 200, and related components.

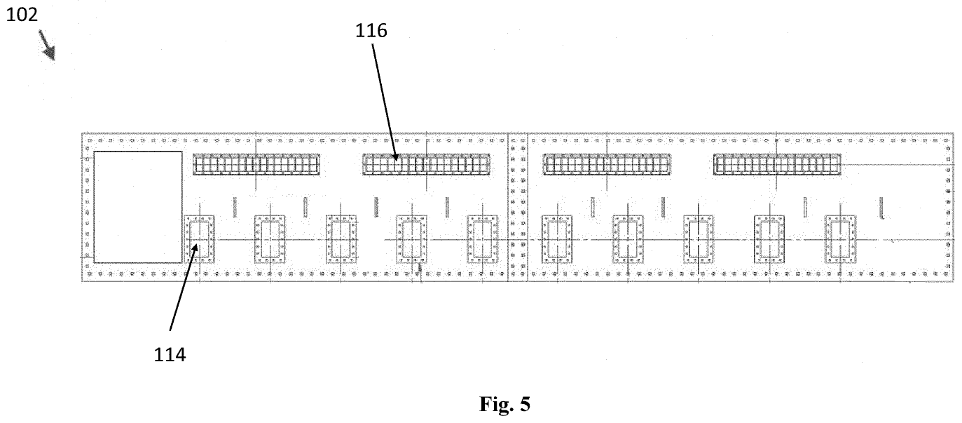

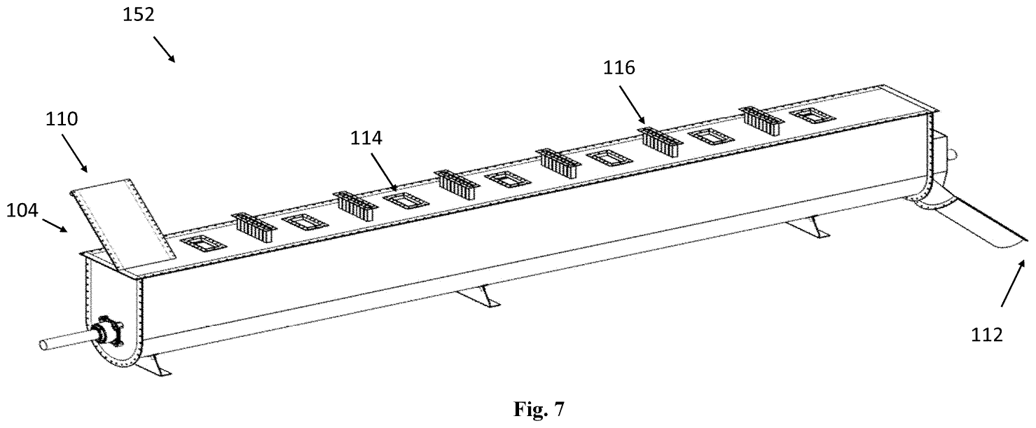

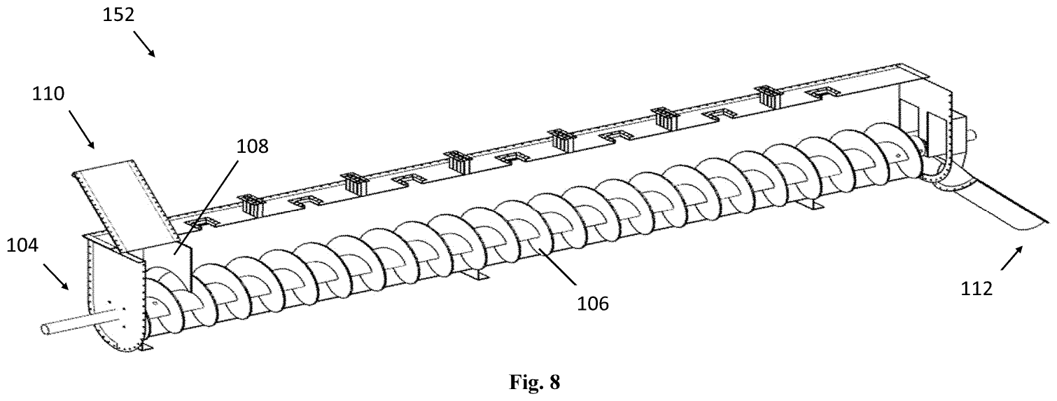

Various example entry points for microwaves via the multiple waveguides 153 in a top of trough 102 are shown in FIG. 5. FIG. 9 shows alternative example entry points in a top of the alternative trough 104. Various other arrangements and configurations of troughs, conveyor units, and/or systems are also contemplated herein. Waveguides 153 are also referred to as microwave guides or simply guides, herein. As shown in FIGS. 7 and 8, the alternative trough 104 can include a material inlet 110 and a material outlet 112. One or both of inlet 110 and outlet 112 can include a microwave suppression tunnel and/or features thereof as described herein.

In the conveyor unit 152 configuration of FIG. 6, the example, alternative trough 104 (or housing) of the continuous heating assembly that includes the auger 106. The auger 106 can optionally be heated and used to cause animal-based material to be heated using liquid and/or microwave heating to be moved longitudinally along the trough 102 of the conveyor unit 152 during material heating, processing, or production. The auger 106 can also be caused to rotate directly or indirectly by the conveyor motor 161 (see, e.g., FIG. 4) (or alternatively, an engine or the like), according to various embodiments. Furthermore, the auger 106 can be caused by the conveyor motor 161 to rotate the auger 106 more slowly or more quickly according to various parameters, which can be based on need or usage, such as target temperature, microwave heating power, and the like. Various controllers can be programmed to rotate the auger 106 according to various set points, parameters, variables, and the like. The motor 161 can have a power rating of 50-150 kW, 70-130 kW, 80-110 kW, or 90-100 kW in various embodiments. Embodiments with the motor having a power rating below 50 kW or above 150 kW are also contemplated.

As shown the auger 106 can be helical, and in some embodiments the auger 106 can be single helical or double helical, among other variations. In yet further variations, a single trough 104 can comprise two separate augers 106, which can be counter-rotating or otherwise (not shown). As shown, a fluid connection can be attached to one or more ends of the auger 106, which can be used for additional auger-based heating or cooling of material being produced.

FIGS. 7-9 show various views of the alternative configuration 104, where various apertures within the alternative trough 104 cover are instead positioned in alternative locations as compared to trough 102. More specifically, the microwave inlets 114 and vents 116 are generally placed in line as shown with trough 104. Various embodiments that utilize trough 104 can be similar to embodiments that utilize trough 102, and various other configurations are also contemplated herein.

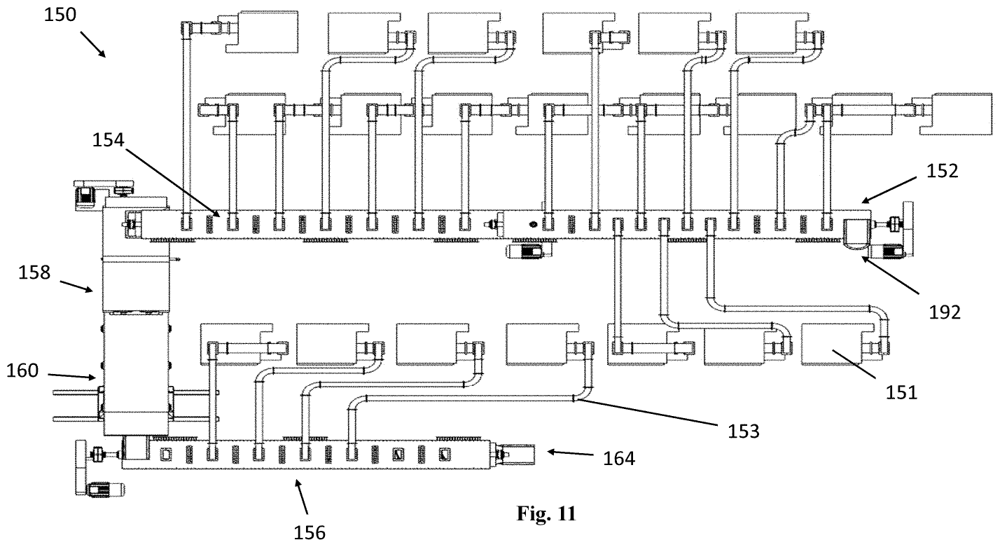

FIGS. 10 and 11 show an example multi-conveyor continuous material treatment or processing system 150. The system 150 as shown comprises an example of three conveyor units that are similar to conveyor unit 152 described above, in addition to a mixer 158, lifting conveyor 160, and two microwave suppression tunnels (e.g., 200, 202) shown at inlet 162 and outlet 164. Multiple microwave heating units 151 are also shown connected to the conveyor units via multiple corresponding waveguides 153 as described herein. As shown the three conveyor units are laid out in series, or sequentially.

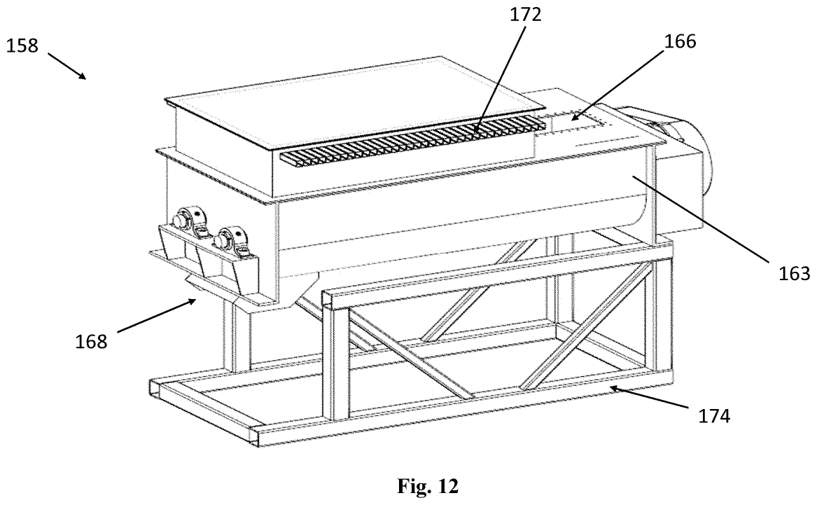

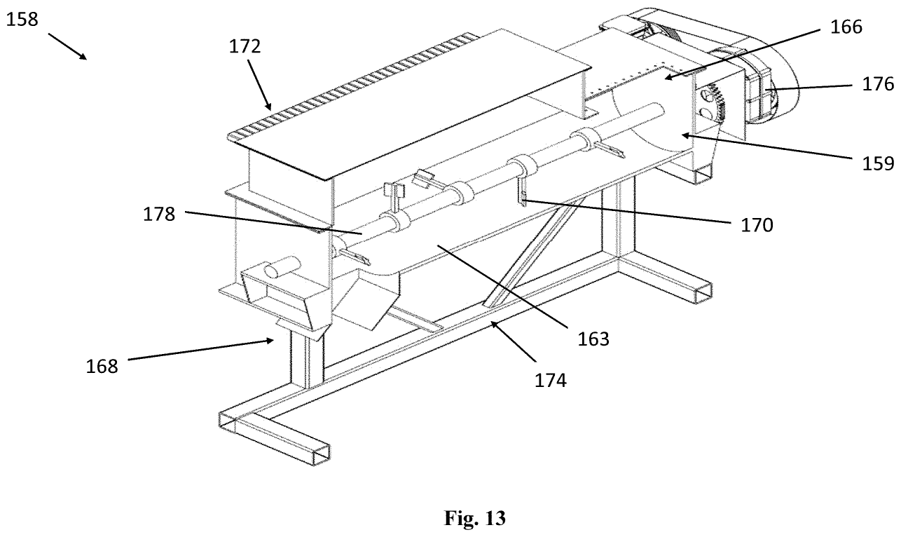

As shown, a first conveyor unit 152 receives animal-based material to be heated, and the system 150 operates sequentially by passing the material to a second conveyor unit 154 following the first conveyor unit 152, and to a third conveyor unit 156 following the second conveyor unit 154. A mixer 158 (described in greater detail with reference to FIGS. 12 and 13), and a lifting conveyor 160 are also shown in line and between the second conveyor unit 154 and the third conveyor 156 in a sequential or serial arrangement. In other optional embodiments, a return system can be implemented where material is returned to the inlet 162 once it has approached or left the outlet 164 or equivalent. In this way, a given system 150 can simulate a larger system and can achieve higher temperatures and/or longer heating times as desired.

In particular, the mixer 158 can be located sequentially after an outlet of the second conveyor unit 154, and the lifting conveyor 160 can be located sequentially after the mixer 158 and before the third conveyor unit 156. The mixer 158 can be a pugmill, a drum mixer, mixing chamber, or any other type of suitable mixer or mechanical processing device as known in the art. The mixer 158 can also be a Brabender type mixer or ball mill, particularly in embodiments where highly-viscous and/or brittle materials are to be combined and/or processed.

As described and shown herein, any number of conveyor units 152, 154, 156, etc. and any number of mixers 158, lifting conveyors 160 can be utilized in various systems such as 150. Moreover, the various components within the system 150 can be arranged in any suitable order according to a desire or need. Furthermore, microwave suppression tunnels (e.g., 200, 202) are preferably utilized at various inlets and/or outlets of the system 150 according to various embodiments.

The various conveyor units 152, 154, 156 can positioned such that the first conveyor unit 152 is vertically elevated and that the second and/or third conveyor units 154, 156 are positioned sequentially lower than the first conveyor unit 152 so as to utilize gravity to facilitate movement of material being heated between the various conveyor units when in use. In some embodiments, one or more mechanical lifting conveyor 160 can also be utilized to lift or raise the material being heated and reduce a total amount of height required for various conveyor units. As used herein, a conveyor, can be any mechanism or setup, or component thereof, that allows or causes a material to be moved from one location to another location.

When used sequentially, the first conveyor unit 152 can heat the flowing material to a first temperature, the second conveyor unit 154 can heat the material to a second temperature greater than the first temperature, and the third conveyor unit 156 can heat the material to a third temperature that is greater than the second temperature according to various embodiments. Each conveyor unit preferably heats the material using microwave energy as the material flows and such that a third or final desired temperature is reached before the material exits the heating and/or processing system, e.g., after achieving a desired heating and time specification per various regulations or desires, and/or chemical reactions, transformations, and/or processing. In various alternative embodiments, each conveyor unit can apply, e.g., a preset amount of energy to the animal-based material, irrespective of temperature levels observed.

Any conveyor unit, such as the first conveyor unit 152, can further include one or more baffle(s) 108 (see FIG. 8), preferably a vertical baffle or a baffle that is otherwise at least partially transverse to a direction of material flow within the conveyor unit 152, which is configured to restrict, guide, and shape the material as it proceeds through the first housing of the first conveyor unit 152. For instance, the baffle 108 can assist the auger 106 in restricting the flow of, leveling the animal-based material to a desired maximum level within the first conveyor unit 152, or reducing the particle or chunk size of received material to a desired diameter and/or flowability for processing and/or heating. In some embodiments, the animal-based material to be processed, before or after passing the baffle 108, has a maximum diameter or size of about eight inches (20.3 cm). In other embodiments the maximum diameter is about six inches (15.2 cm). In yet further embodiments, one or more impactor, shredder, or the like, is added to reduce a maximum largest dimension of the material. For example, in some embodiments at least some material is crushed, comminuted, ground, or otherwise reduced in size, e.g., to be made flowable, within or prior to entering the first conveyor unit 152. For example, various received animal-based materials may contain relatively rigid and bulky component parts, such as bones or other substances either originally part of an animal or otherwise. Other conveyor units can also include various types of baffles (e.g., baffle 108) or other restrictive or material guiding members or features. In other embodiments, the material is received as a semi-solid, liquid, or flowable state. During heating the material can progressively become more solid and less flowable as water is evaporated or boiled off the material. In other cases, the material can become more flowable as water is boiled off.

FIGS. 12 and 13 show the optional mechanical processing mixer 158 of system 150 in greater detail. The mixer 158 generally includes a mixer trough 163 supported by a mixer support structure 174, which can be height-adjustable in various embodiments. The mixer 158 also preferably comprises one or more mixer vents 172, and a mixer material inlet 166 and outlet 168. With reference in particular to the cross-sectional view of the mixer 158 in FIG. 13, the mixer trough 163 has an interior 159 for holding and mixing a material being processed. The mixer trough 163 also supports a mixer shaft 178 (e.g., via one or more bearings, not shown) that is operatively driven by a mixer motor 176. Connected to and protruding from the mixer shaft 178 are one or more mixer axially-mounted paddles 170 that are configured to mix a material held within the interior 159 of the mixer trough 163. Optionally, various heat exchanger components and/or heat recovery components or features can be positioned within or near the mixer 158. As shown the material is not heated during mixing within mixer 158. However, in alternative embodiments, the material can be heated while in the mixer 158. Multiple mixer shafts 178 can optionally be included in mixer 158.

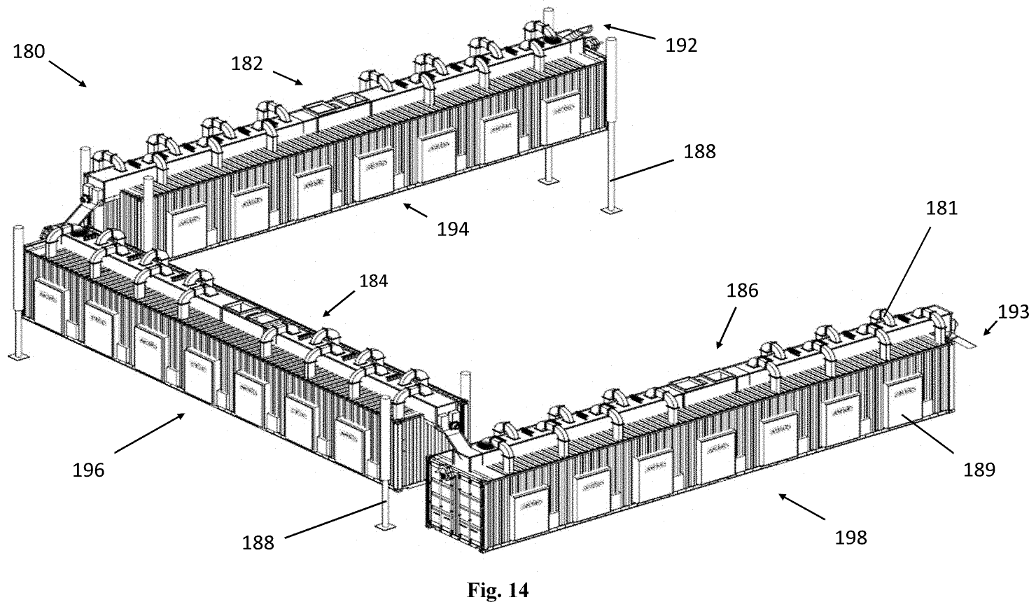

FIGS. 14 and 15 show various mobile multi-conveyor continuous treatment systems, including 180 (three conveyor unit) and 190 (two conveyor unit).

Mobile and/or modular multi-conveyor continuous treatment systems, such as systems 180 or 190, can be beneficially modular and easily transported. With mobile, modular systems, scalability of production can be improved because additional mobile units can be added for a jobsite as needed, provided there is sufficient space, and without requiring additional fabrication or sourcing of components.

As shown in FIG. 14, a three-module, mobile multi-conveyor mixer and treatment system 180 is shown. The system 180 as shown is composed of three generally similar mobile container units 194, 196, and 198, each comprising a conveyor unit 182, 184, and 186, respectively. As shown, each mobile container unit also comprises one or more microwave units 189, one or more waveguides 181, and optionally one or more system material inlet 192 and/or outlet 193. According to some embodiments, each mobile container unit 194, 196, and/or 198 is one or more reused or modified industry standard corrugated steel shipping container. Various openings and/or portions can be removed or modified such that the various components can fit onto or within each mobile container unit. As shown, the conveyor units 182, 184, 186 are generally positioned above or on an upper portion of the respective mobile container unit 194, 196, 198. The microwave heating or power units 189 are shown as being at least partially integrated into the mobile container units 194, 196, 198, and at least a portion of each microwave heating unit 189 can be exposed to the outside when installed within the mobile container unit. Various container units 194, 196, 198 as contemplated herein can be mounted to or incorporated various vehicles, trailers, etc.

Each mobile container unit 194, 196, 198 can further be provided with a mechanism or system for adjusting a vertical position or height of the mobile container unit operative components, such as the conveyor unit and/or various mechanical material processing units. The mechanism can include one or more individual adjustable height support structures 188, e.g., four with one positioned at each corner of each mobile container unit. Other height-adjustable structures are also contemplated, such as various scissor lifts, jacks, removable stands, and the like.

As shown the first mobile container unit 194 is positioned at a relatively more raised position, the second mobile container unit 196 is positioned at a less raised position compared to the first mobile container unit 194, and the third mobile container unit 198 is positioned at a fully lowered position, e.g., set on a ground or floor without use of the adjustable height support structures 188. Although neither a mixer (e.g., 158) nor a lifting conveyor (e.g., 160) are shown in the system 180, in other embodiments one or more mixers and/or lifting conveyors can be utilized with the system 180, and can be integrated into one or more mobile container units, such as 194, 196, and/or 198. Any feature or component of system 150 of FIG. 14 can be applied to the system 18, as appropriate. As discussed herein, the mixer 158 can be replaced or supplemented by any suitable processing unit, including any mechanical material processing unit.

FIG. 15 shows an alternative mobile multi-conveyor material mixer and treatment system 190 with a single combined mobile container unit 199 with two conveyor units 182, 184 therein. As shown, a single container, such as a shipping container, can be modified to receive two conveyor units 182, 184 in sequence, and optionally can include a mixing and/or venting chamber 183 positioned between the first and second conveyor units 182, 184. Multiple systems 190 can be operated in parallel in order to adjust a throughput of heated material according to a particular need or desire for a mobile operation.

FIGS. 16-31 illustrate various arrangements of features of microwave suppression tunnels or chutes, such as the inlet suppression tunnel 202 or the outlet suppression tunnel 200. As used herein, the inlet suppression tunnel 202 and the outlet suppression tunnel 200 can be operatively similar and the features of either can be incorporated into the other in various embodiments. For example, although the inlet suppression tunnel 202 is shown with a single flap 218, multiple flaps 218 can be used in the inlet suppression tunnel 202 among other features of the outlet suppression tunnel 200. For example, the various suppression tunnels of FIGS. 16-31 can be adapted to connect and operate in conjunction with systems 150, 180, and any other system disclosed herein, among other examples.

As shown in FIG. 16, the outlet suppression tunnel 200 can be configured to include one or more absorbing, deflecting, or blocking flaps 214, variously including inlet and outlet suppression tunnel embodiments. Each suppression tunnel can be located attached to or comprised within a material inlet (e.g., inlet suppression tunnel 202) or outlet (e.g., outlet suppression tunnel 200) of various conveyor units as described herein. The example outlet suppression tunnel 200 preferably comprises a chute flange 207 for attachment at or near a conveyor unit outlet, or the like. The suppression tunnel 200 can also be configured for use as an “inlet” suppression tunnel with only minor changes, such as changing the location of the chute flange 207, a direction of permitted flap 214 movement relative to the outlet suppression tunnel 200, positioning, and the like. The flap 214 can be a single unit that is movable, flexible, or the like as described below. Flap 214 is attachable and/or pivotably attached to an upper portion of the outlet suppression tunnel 200.

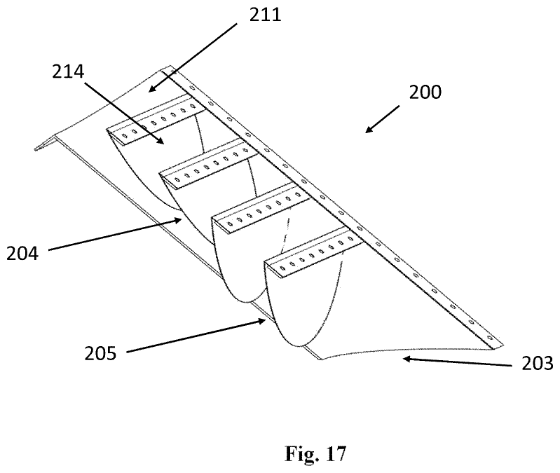

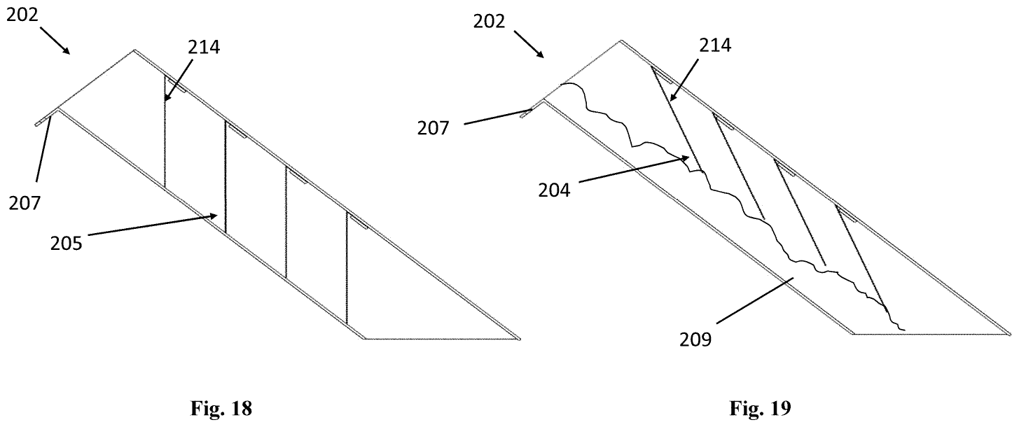

Shown in perspective cross-sectional view in FIG. 17, the outlet suppression tunnel 200 includes flaps 214 that can move from a default, closed position 205 of the flap 214 as it contacts the outlet suppression tunnel 200, to a dynamic, open position 204 as material 209 flows past (see FIG. 19), and applies a pressure on the flap 214, thereby opening it until the material 209 stops flowing or is cleared from the outlet suppression tunnel 200 (See FIG. 18). The outlet suppression tunnel 200 as shown in FIGS. 16 and 17 includes an attachment side, tunnel inlet 211, and an exit side, tunnel outlet 203.

An alternative embodiment of a flap 220 for use herein, is instead composed of multiple sub-portions 222, such as strips of microwave blocking, deflecting, or absorbing material, which are attached to an attachment flange 224 of the flap, which is usable for attachment (e.g., pivotable attachment) of flap 220 to an upper portion of the suppression tunnel 220. In yet further alternative embodiments of suppression flaps, chains, combinations of materials, or any other suitable microwave-suppression composition can be utilized.

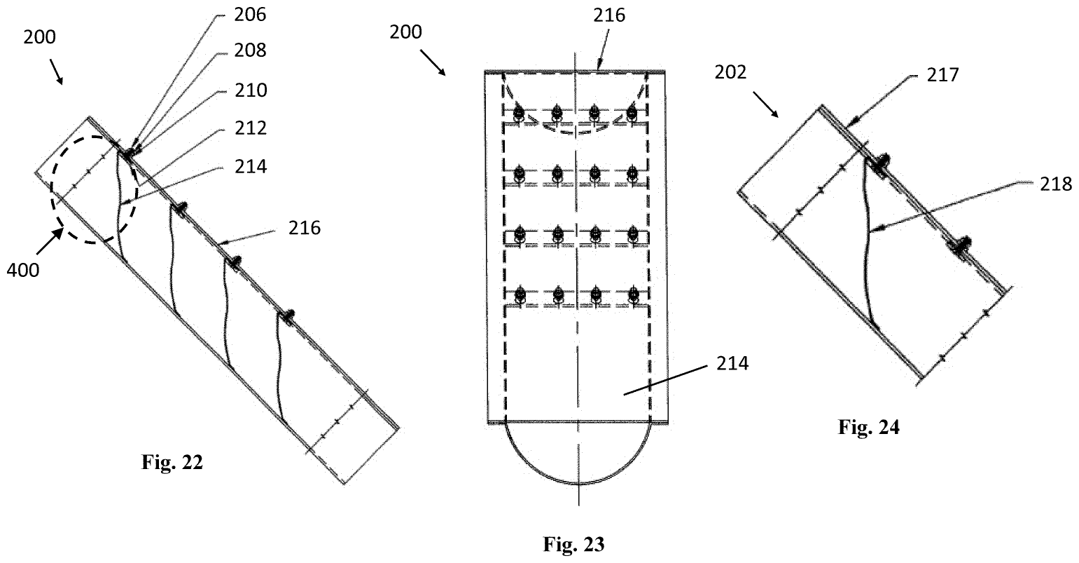

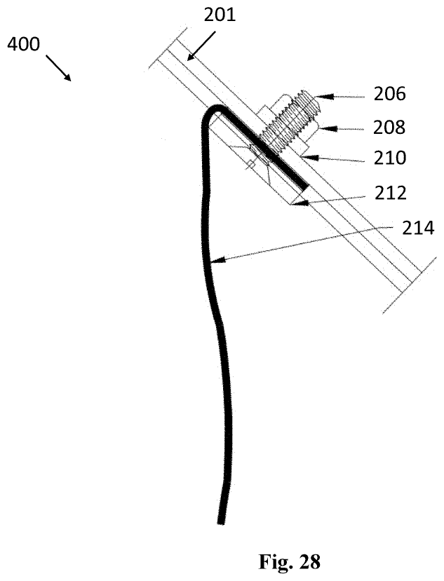

FIG. 22 is a cross-sectional side view of a U-shaped outlet suppression tunnel 200 of an outlet side. As shown, a series of four, single-ply (e.g., single layer) microwave suppression flaps 214 are shown in the outlet suppression tunnel 200 in a down position. At hardware detail section 400 of FIG. 28, flaps 214 can be attached to a top outlet side portion 216 of the outlet suppression tunnel 200 along with attachment hardware including bolt fastener 206, nut 208, bolt washer 210, metal bracket 212, and shielding mesh flap 214.

FIG. 23 is a cross-sectional top view of the outlet U-shaped microwave outlet suppression tunnel 200 of FIG. 22. As shown, multiple attachment points (e.g., using hardware shown at FIG. 28) for each flap 214 are contemplated, although any suitable attachment or arrangement for the flap 214 is also contemplated herein.

FIG. 24 is a cross-sectional side view of a U-shaped inlet microwave suppression tunnel 202 for use with or connection to an inlet side of a conveyor unit, such as conveyor unit 152 of the system 100. System 100 described above with reference in particular to FIGS. 1-4 can have inlet and outlet ends of a continuous motion particle pathway (e.g., motivated by auger 106 or other conveyance mechanism of the conveyor unit 152), an inlet suppression tunnel 202 can be used with or without an outlet suppression tunnel 200 as shown in FIGS. 22 and 23. A single, single-ply (e.g. single layer) microwave suppression flap 218 is shown in FIG. 24 attached to a top inlet side portion 217, e.g., using hardware as shown and described with respect to FIG. 28, below. As shown in the embodiments of FIGS. 22-24, the outlet/inlet suppression tunnels 200 and 202 use a single-ply (e.g., single layer) microwave-absorbing, deflecting, or blocking mesh flap 214 or 218, respectively. With reference to mesh flaps 214 and 218 and the like, the term “absorbing” is understood generally to optionally include any of absorbing, deflecting, blocking, and/or any other suppression technique of microwaves.

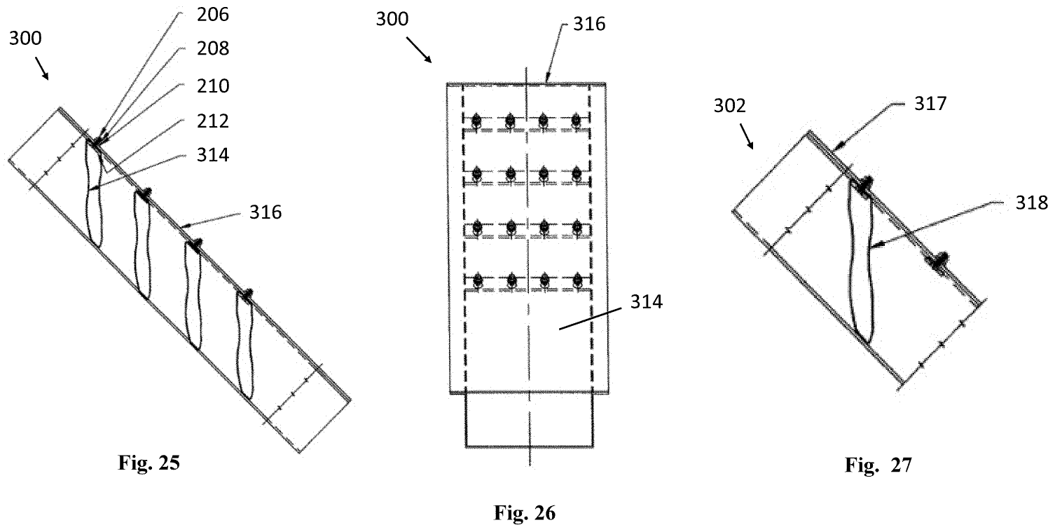

FIGS. 25-27 illustrate alternative embodiments where mesh flap(s) 314, 318 are doubled over as two-ply for increased microwave absorption. FIGS. 25-27 are similar to FIGS. 22-24, respectively, with the exception of the folded over, two-ply (two layer) mesh flap(s) 314, 318.

FIG. 25 is a cross-sectional side view of a rectangular microwave outlet suppression tunnel 300. Four flaps 314 are shown, and each flap 314 can be attached to a top portion 316 of the outlet suppression tunnel 300 along with attachment hardware including bolt fastener 206, nut 208, bolt washer 210, metal bracket 212, and shielding mesh flap 314.

FIG. 26 is a cross-sectional top view of the rectangular microwave outlet suppression tunnel 300 of FIG. 25. FIG. 27 is a cross-sectional side view of a corresponding rectangular microwave inlet suppression tunnel 302. As shown, folded flap 318 is attached to top outlet side 317.

FIG. 28 shows greater detail of hardware detail section 400 of FIG. 22. As shown, a flap 214 can be attached to (e.g., a top inlet or outlet side portion) of a suppression tunnel along with attachment hardware including bolt fastener 206, nut 208, bolt washer 210, metal bracket 212, and shielding mesh flap 214. FIG. 28 shows a side view of a non-looped, single-ply microwave absorbing, deflecting, or blocking flap 214 with a microwave-absorbing, deflecting, or blocking mesh described in greater detail herein that is attached to an upper portion of a suppression tunnel (or chute thereof, etc.). Only one example fastening arrangement is shown at hardware detail section 400, but other arrangements are contemplated. In other embodiments, the flap 214 with mesh can be looped, causing a two-ply (e.g., two layer) flap to be attached at two ends in a manner similar to the fastening arrangement shown at hardware detail section 400.

Flap 214 as shown in FIG. 28 (and any other embodiments of flaps herein) is preferably electrically grounded to a heating system frame 201. The heating system frame 201 is preferably grounded to a power source electrical grid (not shown) according to various embodiments.

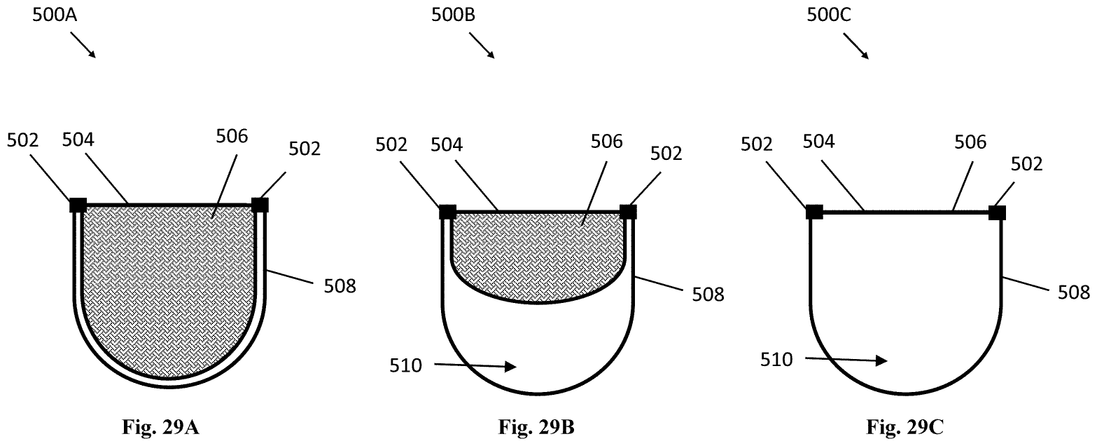

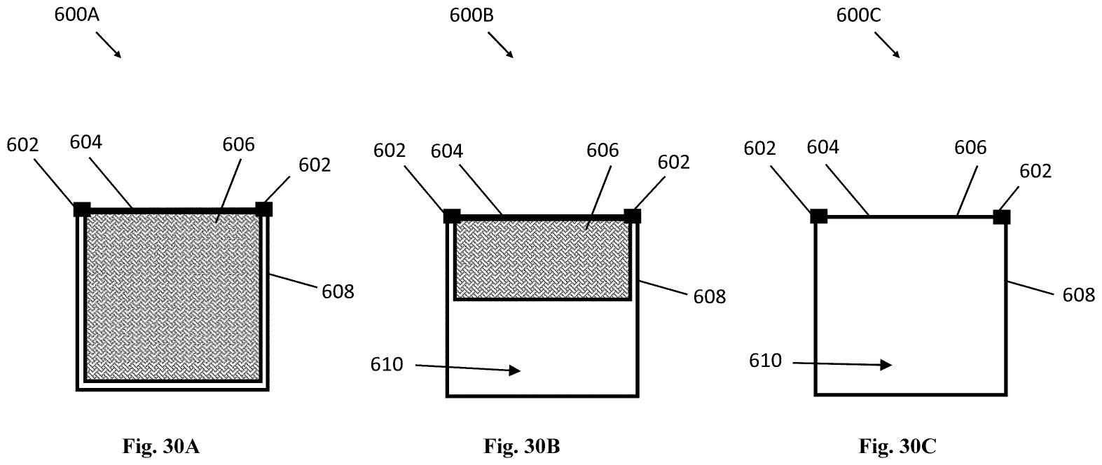

Turning now to FIGS. 29A-29C and 30A-30C, various cross-sectional end views are shown that provide detail of flap configuration within a suppression tunnel or chute in addition to flap articulation or flexing that occurs during continuous animal-based material processing, production, and movement along the tunnel.

Inlet and/or outlet microwave suppression tunnels (e.g., 202, 200, etc.) can be positioned and connected relative to the continuous heating assembly or system as described herein. During heating operation, it is possible that at least some microwave energy will not be absorbed by material being heated or other components within the assembly. This non-absorbed, escaped, or “leaked,” microwave energy can be unsafe, undesirable, or otherwise beneficial to avoid in practice. In order to address this shortcoming, one or more movable and/or pivotable flaps can be positioned at the inlet tunnel, the outlet tunnel, or both.

In various embodiments, an example microwave absorbing, deflecting, or blocking flap, for inlet or outlet of material, can comprise a flexible mesh configured to freely pivot when contacted by moving animal-based material as described herein. Inlet and/or outlet microwave suppression tunnels can have rounded, rectilinear, or a combination of the two for an outline along the various tunnels.

In various embodiments, the various microwave suppression tunnels are preferably in a substantially horizontal position, but preferably at an angle of no more than 45 degrees from horizontal.

FIG. 29A is a cross-sectional end view of a U-shaped microwave suppression tunnel configuration 500A with a top-mounted pivoting mesh flap 506 in a closed position. Example attachment points 502 show one alternative mounting configuration that allows flap 506 to pivot within U-shaped flap surround 508. The flap 506 can pivot along a top flap portion or axis 504, or can bend alternatively when a pressure is applied to the flap 506.

FIG. 29B is a cross-sectional end view of a U-shaped microwave suppression tunnel configuration 500B, similar to 500A of FIG. 29A with the mesh flap 506 in a partially open position. As particles are moved along a trough defined by surround 508, flap 506 can be caused to pivot or bend such that an opening 510 between the flap 506 and the surround 508 is revealed. Opening 510 can allow material particles to pass while allowing minimal microwaves to escape. Particles of material causing flap 506 to open can at least partially block microwaves that would otherwise have escaped the microwave suppression tunnel (e.g., outlet suppression tunnel 200 or inlet suppression tunnel 202, among other examples described herein).

FIG. 29C is a cross-sectional end view of the U-shaped microwave suppression tunnel configuration 500C similar to 500A of FIG. 29A with the mesh flap 506 in a fully open position, causing a larger opening 510 than in configuration 500B.

The embodiments shown in FIGS. 29A-29C can also be modified to include a rectangular flap 606 with a corresponding rectangular tunnel or chute surround 608, as shown in FIGS. 30A-30C.

FIG. 30A is a cross-sectional end view of a rectangular microwave suppression tunnel configuration 600A with a top-mounted pivoting mesh flap 606 in a closed position. Example attachment points 602 show one alternative mounting configuration that allows flap 606 to pivot within rectangular flap surround 608. The flap 606 can pivot along a top flap portion or axis 604, or can bend alternatively when a pressure is applied to the flap 606.

FIG. 30B is a cross-sectional end view of a rectangular microwave suppression tunnel configuration 600B, similar to 600A of FIG. 30A with the mesh flap in a partially open position. As animal-based material particles are moved along a trough defined by surround 608, flap 606 can be caused to pivot or bend such that an opening 610 between the flap 606 and the surround 608 is revealed. Opening 610 can allow particles to pass while allowing minimal microwaves to escape. Material particles causing flap 606 to open can at least partially block microwaves that would otherwise have escaped the microwave suppression tunnel.

FIG. 30C is a cross-sectional end view of the rectangular microwave suppression tunnel configuration 600C similar to 600A of FIG. 30A with the mesh flap 606 in a fully open position, causing a larger opening 610 than in configuration 600B.

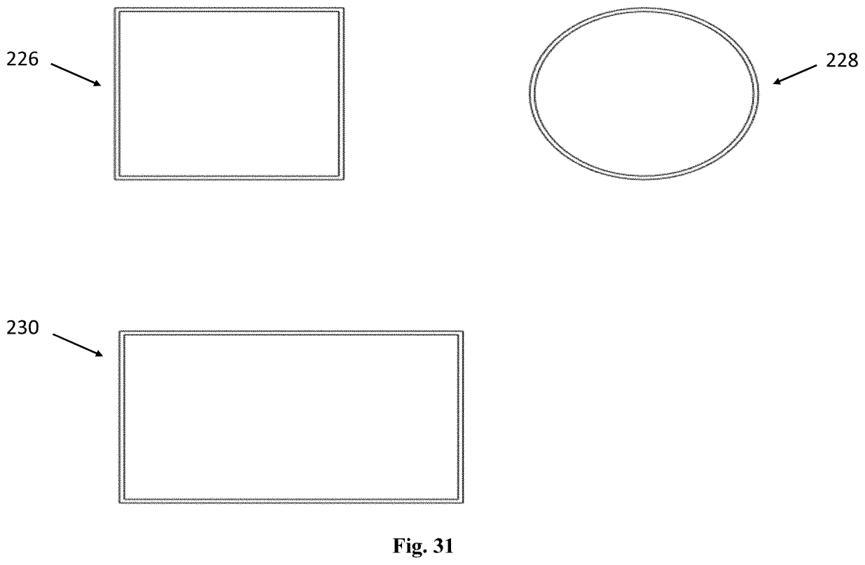

Many other microwave suppression system flap and tunnel configurations are also contemplated herein, and the examples above are merely shown as selected examples of preferred embodiments. For example, various example and alternative cross-section shapes of chute are shown at FIG. 31. A generally square chute cross-section is shown at 226, a generally round chute cross-section is shown at 228, and a generally rectangular chute is shown at 230. Any other shape of chute or suppression tunnel (and correspondingly shaped flap[s]) is also contemplated herein.

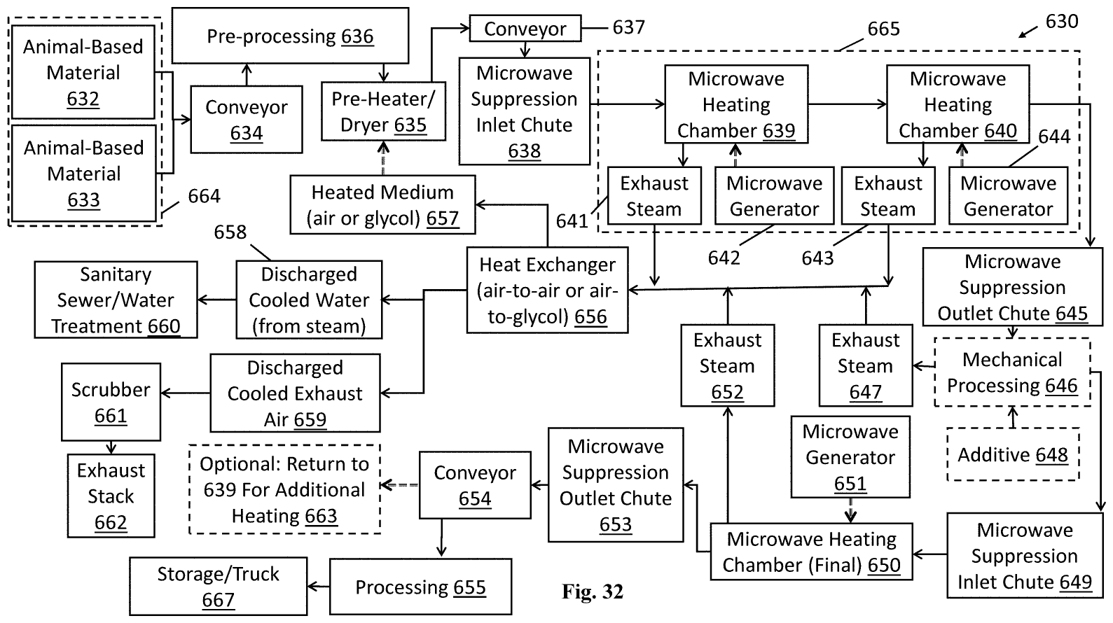

FIG. 32 is a flowchart of an example process 630 according to embodiments of the present disclosure.

Process 630 can start with operations 632 and/or 633. At operation 632, one or more hoppers (e.g., containers) of animal-based material are optionally weighed. At operation 633, one or more hoppers (e.g., containers) of animal-based material are also optionally weighed. As shown at 664, multiple bins, containers, piles, silos, or the like of various animal-based materials 632, 633 can be combined with different materials (or in some cases, combined with other non-animal-based materials) to obtain an animal-based material blend. The optional material blend is referred to as animal-based material below for simplicity. For example, certain types of animal-based materials may be mixed in small quantities to another material for processing according to various properties.

Next, process 630 proceeds to operation 634, where a conveyor (e.g., a loader unit) carries animal-based material to a pre-heater or drier at 635. Optionally at operation 636, a moisture/water content of the material can be determined or an average moisture content level for the type of material can be estimated and entered. By determining an initial moisture content, the initial weight of the animal-based material can be used to predict or determine final dry weight and the mass of water to be removed. Also at 635, energy can be transferred to the pre-heated or dryer from a heated medium, such as air or glycol from operation 657, as discussed further below.

Following operation 635, the animal-based material can be further moved using another conveyor at operation 637 until the material reaches a microwave suppression inlet chute (or tunnel) at operation 638. Next, the material can proceed to a microwave heating chamber (e.g., a trough of a conveyor unit), which can emit heated exhaust steam at 641, and can receive power via microwaves emitted by a microwave generator at 642 (e.g., via one or more waveguides as discussed herein).

Optionally, the material can then proceed to another microwave heating chamber of another conveyor unit at 640, which can also omit exhaust steam at 643 and/or receive microwave energy from another microwave generator at 644 (e.g., a microwave heating unit, etc.). As shown at 665, multiple heating sections can be added to get the required energy input to reach a specific throughput and/or reach a specification, such as according to a regulation or desired characteristic. After the animal-based material is sufficiently heated in accordance with desired specifications, the material can proceed to as past a microwave suppression outlet chute (or tunnel) at 645.

After the material passes the microwave suppression outlet chute at 645, optionally the material can enter an agitator/mixer or any other mechanical processor at 646. The material when in the mixer (if present) can emit exhaust steam at 647, and can optionally receive an additive (e.g., to make a final product more suitable for use as fertilizer, cow bedding, etc.) at 648. It is contemplated that in some embodiments no mixer 646 is used, and the microwave heating chamber 640 can proceed to microwave heating chamber 650 without a mixer.

If the mixer 646 is used, and once the material is sufficiently mixed at 646, the material can proceed to another microwave suppression inlet chute (or tunnel) at 649.

At 650 (and similar to 639 and 640), the material can proceed to a third microwave heating chamber at 650. The chamber 650 can also receive microwave energy via one or more microwave generator at 651, and exhaust steam can also be used to extract heat from the heated material at 652. Once the material is heated to a desired, final temperature and moisture and microbial content level at 650, the material can proceed through another microwave suppression outlet chute at 653, and can proceed via a conveyor 654 (e.g., now as an animal product) to a storage medium, such as a silo or shipping truck/vessel/train at 667, among other destinations for storage or use, including at various remote locations. Optionally before storage at 667, the material or product can be subject to one or more additional processing operations at 655. If, however, the material may benefit from additional heating and/or drying, at 663, the material being processed can be returned to, e.g., microwave heating chamber 639 (e.g., via microwave suppression inlet chute 638) for additional processing. Material can be returned for additional processing two, three, four or any number of times and suitable based on target specifications of the processed animal-based material.

Exhaust steam heat received at 641, 643, and/or 652 can be recovered as waste heat using one or more heat exchanger 656. The heat exchanger 656 can be an air-to-air heat exchanger, or an air-to-liquid (e.g., glycol) heat exchanger in various embodiments. The heat exchanger 656 can thereafter provide heat via a heated medium at 657 to be used in the pre-heater or dryer 635 as discussed above.

Also, in thermal communication with the heat exchanger at 656 can be discharged cooled water (from steam) at 658 and/or discharged cooled exhaust air at 659. The discharged cooled water at 658 can then proceed to a sanitary sewer or water treatment at 660. Furthermore, the discharged cooled exhaust air at 659 can proceed to an optional scrubber at 661, and then to one or more exhaust stacks at 662. The optional scrubber at 661 can condense steam and reduce odor emissions and the like.

In some examples, a shielding mesh used for blocking or absorbing microwave emissions can be an aluminum and steel mesh with a pitch or opening size of about 0.15″ (3.81 mm) or less. The shielding mesh can be optionally encapsulated or coated in a protective substance, such as silicone or the like. In some embodiments, such silicone can reduce the likelihood of screens touching and resulting arcing. Reducing arcing between screens can prolong useful life of the screen. Also contemplated is an aluminum particle filled silicone structure. Other variations and types of shielding mesh also contemplated are discussed below.

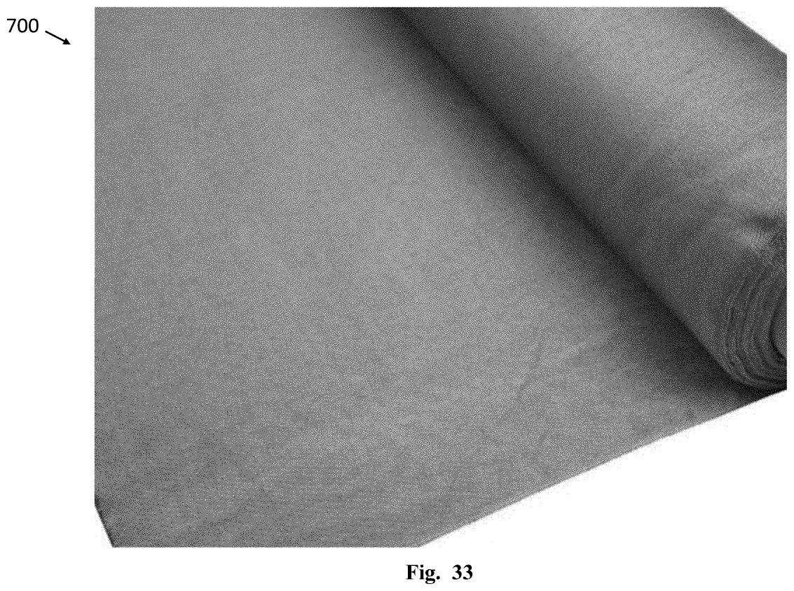

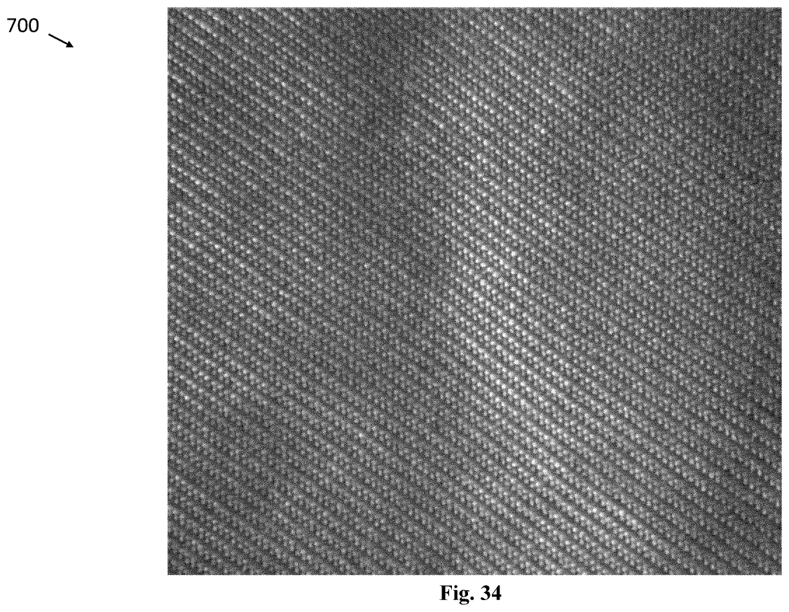

FIGS. 33 and 34 show an example stainless steel RFI shielding mesh 700. The mesh 700 can be a carbon cover metal.

For example, the shielding mesh 700 can be sourced from Aaronia USA/Aaronia AG. The shielding mesh 700 can be an 80 dB Stainless Steel RFI Shielding Aaronia X-Steel model, which can provide military or industrial grade screening to meet various demanding usage cases. In some examples, the shielding mesh 700 can be coated with a polytetrafluoroethylene (i.e., PTFE or “Teflon”) coating, silicone, polyurethane, plastic, or the like.

The steel mesh 700 can be highly durable, effective up to about 600° C., operate under a very high frequency range, and be permeable to air. In more detail, shielding mesh 700 is an Aaronia X-Steel component that can operate to at least partially shield both radio frequency (RF) and low frequency (LF) electric fields.

Some specifications of the shielding mesh 700 can include a frequency range of 1 MHz to 50 GHz, a damping in decibels (dB) of 80 dB, a shielding material including stainless steel, a carrier material including stainless steel, a color of stainless steel (silver), a width of 0.25 m or 1 m or some variation, a thickness of about 1 mm, available sizes of about 0.25 m² or 1 m², a mesh size of approximately 0.1 mm (multiple ply/layer), and a weight of approximately 1000 g/m². The shielding mesh 700 can be suitably durable, and can be configured and rated for use in industrial or other applications, can have a temperature range up to 600° C., can be permeable to air, and permit very easy handling.

In some examples, the shielding mesh 700 can be electromagnetic compatibility (EMC) screening Aaronia X-Steel from Aaronia AG, which can be made from 100% stainless steel fiber. The shielding mesh 700 can meet various industrial or military standards. The shielding mesh 700 can be very temperature stable for at least 600° C., does not rot, is permeable to air. The shielding mesh 700 can be suitable for EMC screening of air entrances and can be very high protective EMC clothing, etc. The shielding mesh 700 can protect against many kinds of RF fields and can offer a 1000-fold better shielding-performance and protection especially in the very high GHz range as compared to various other types of shielding mesh. The shielding mesh 700 provides high screening within the air permeable EMC screening materials. Application examples of the shielding mesh 700 include: Radio & TV, TETRA, ISM434, LTE800, ISM868, GSM900, GSM1800, GSM1900, DECT, UMTS, WLAN, etc.

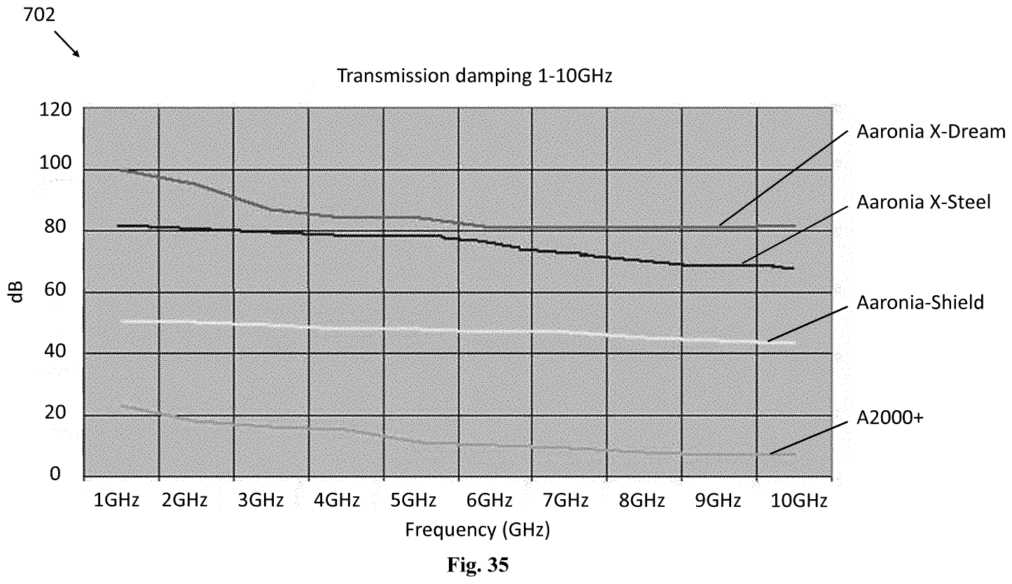

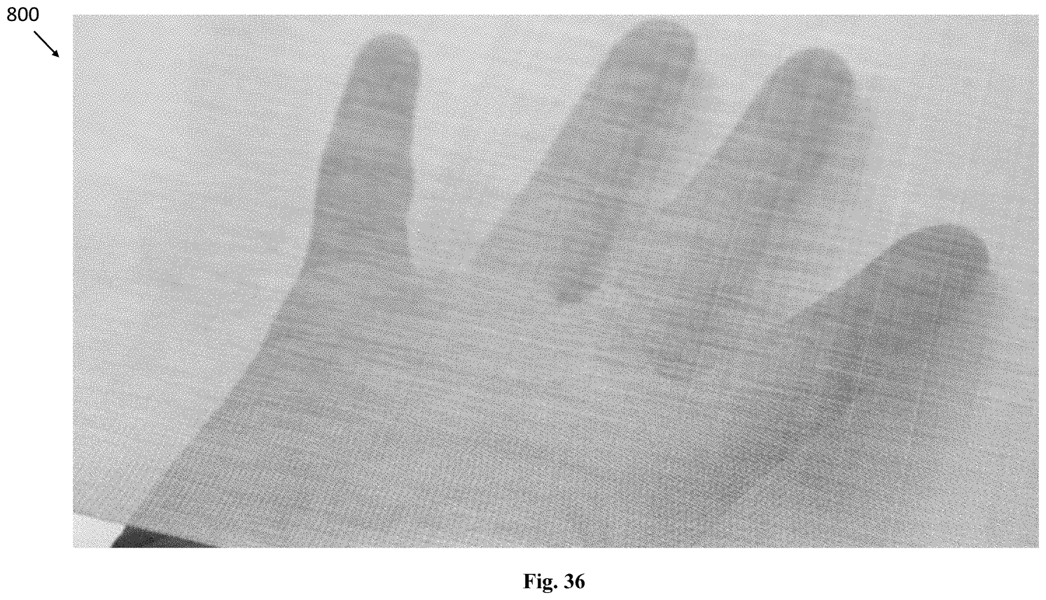

FIG. 35 shows a transmission damping chart 702 for various shielding mesh examples from 1-10 GHz in terms of dB for the mesh 700 of FIGS. 33 and 34. As shown, four shielding meshes are depicted. As shown, in descending order for transmission damping across 1-10 GHz, are Aaronia X-Dream, Aaronia X-Steel, Aaronia-Shield, and A2000+.

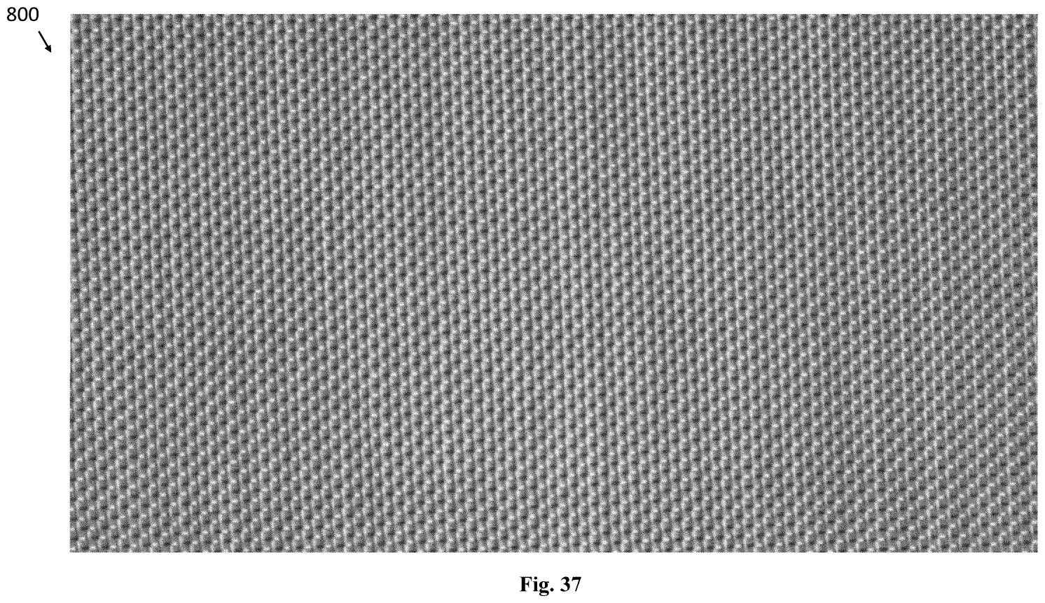

FIGS. 36 and 37 show another example shielding mesh, a fireproof shielding fabric mesh 800.

The fireproof shielding fabric mesh 800 can be sourced from Aaronia AG, and is a stainless-steel EMC/EMF shielding mesh for usage under extreme conditions. The fireproof shielding mesh 800 is usable up to 1200° C., can be half transparent, has high attenuation, and is both odorless and rot resistant. The fireproof shielding fabric mesh 800 has microwave attenuation as follows: 108 dB at 1 kHz, 100 dB at 1 MHz, 60 dB at 100 MHz, 44 dB at 1 GHz, 30 dB at 10 GHz.

Some specifications of the fireproof shielding fabric mesh 800 include: lane Width: 1 m; thickness: 0.2 mm; mesh size: about 0.1 mm; color: stainless steel; weight: approx. 400 g/m; usable until about 1200° C.; yield strength: 220 MPa; tensile strength: 550 MPa; hardness: 180HB; can be breathable; odorless; transparent; rot resistant; frost proof; washable; foldable; bendable; mesh material: stainless steel.

The fireproof shielding fabric mesh 800 has screening performance for static fields of: 99.9999% to 99.99999% (e.g., when grounded). The fireproof shielding fabric mesh 800 has screening performance for low electric fields of: 99.9999% to 99.99999% (e.g., when grounded).

The fireproof shielding fabric mesh 800 is suitable for industrial applications as well as for research and development. The fireproof shielding fabric mesh 800 has been specifically designed for use under adverse conditions (salt air, extreme temperatures, vacuum, etc.).

The fireproof shielding fabric mesh 800 is made of 100% stainless steel, is temperature stable up to 1200° C., has a high microwave attenuation, and yet is breathable. The material of mesh 800 absorbs reliable E&H fields. In particular, in the kHz and low MHz range mesh 800 offers a high shielding factor of up to 108 dB (E-field). Mesh 800 is easy to process and can be cut with a standard pair of scissors.

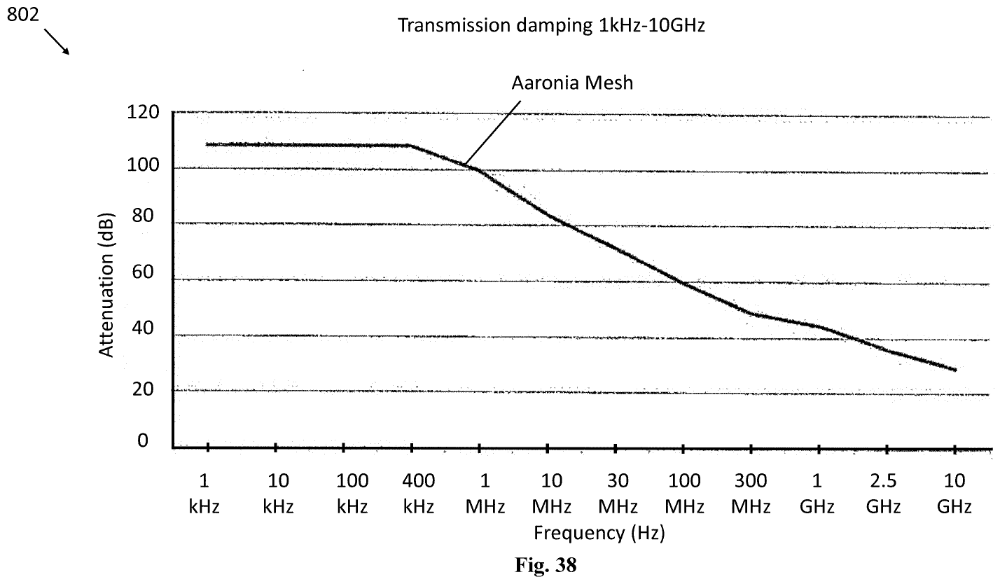

FIG. 38 is a transmission damping chart 802 from 1-10 GHz in terms of dB for the fireproof mesh 800 of FIGS. 36 and 37.

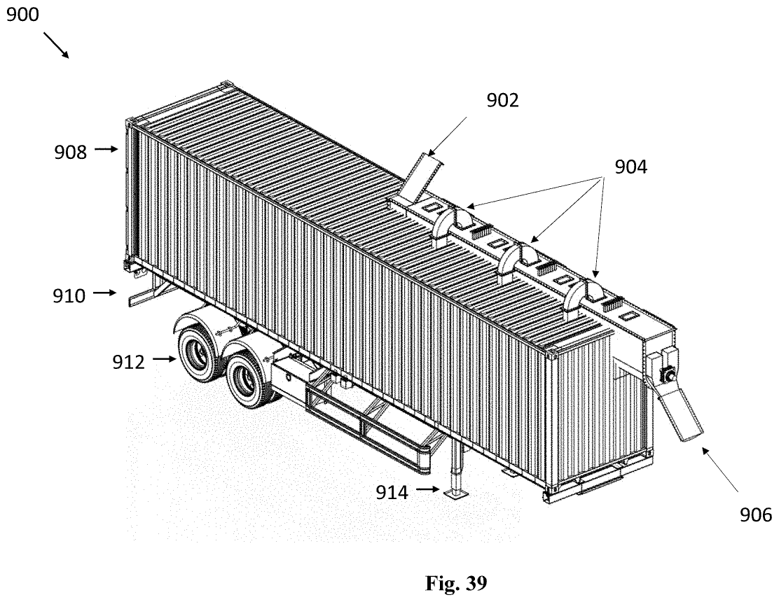

FIG. 39 is a perspective view of another embodiment of a portable, continuous microwave animal-based material processing system 900. The system 900 includes a trailer 910 with wheels 912, and a body 908. The body 908 is preferably supported by the trailer 910 and can be removable therefrom in some embodiments. The body 908 can be a shipping container or a modified shipping container in various embodiments. As described in other embodiments herein, the system 900 includes an inlet 902, one or more microwave waveguides 904, and an outlet 906, in addition to preferably including one or more microwave generators (not shown) internally to the body 908. The trailer 910 is also equipped optionally with one or more stabilizers 914, which can be used for leveling the system 900 when a tractor or truck (not shown) is removed from the trailer 910. The stabilizers 914 can be telescopic and adjustable in length. The system 900 is preferably substantially level when prepared for material heating operation. As the system 900 is portable and/or towable, it is easily transported between various animal processing sites, farms, stockpiles, and/or facilities. Smaller and/or scaled down versions of the system 900 can meet certain target temperatures and heating times according to certain physical and mechanical limitations and constraints.

With reference to portable systems such as 900, in some embodiments a municipality or facility can be equipped with an auger configured to deliver material from a centrifuge. In some cases, a clearance height of the auger can be insufficient to get the system 900 unit under the auger. An additional conveyor can in such cases be implemented to bridge a gap or otherwise connect a facility to the system 900. Therefore, it is contemplated that some additional form of material handling equipment can be used to adapt the system 900 to an existing system or facility.

In preferable embodiments, and in particular where ambient temperatures are relatively low, the conveyor unit can be thermally insulated to better maintain heat, which can increase efficiency significantly. Steam heat exchangers may be well-suited for implementation so as to improve overall system efficiency. Improvements to efficiency are desirable for many reasons. For example, a more efficient system can handle larger volumes of animal-based materials, and can decrease pasteurization time due to higher resulting temperatures, or alternatively can use less power to obtain the same heating rate.

The fatty nature of certain animal-based materials, such as animal sewage and body-derived products, can make the materials sticky with respect to the conveyor unit housing, e.g., steel. A non-stick coating such as Teflon can therefore be beneficially applied to the conveyor unit case to reduce or prevent the sticking of the materials. In addition, or in the alternative, side walls of the conveyor unit housing can be cleaned continuously or periodically according to various embodiments.

As disclosed herein, sterilizing of animal-based materials is by microwave radiation. In some embodiments, after said sterilizing, no single viable microbial species is present in amounts in excess of about 50 (colony forming unit) cfu/g. In some embodiments, after said sterilizing, no single microbial species is present in amounts in excess of about 10 cfu/g. In some embodiments, said sterilizing is by heating at a temperature of from about 120° C. to about 160° C. In some embodiments, the residence time (calculated merely by measuring the time that the solid protein feed product is exposed to inactivation conditions) during said sterilizing is less than about 20 minutes.

As discussed above, and in some embodiments, said sterilizing is by microwave radiation. In some embodiments, a wavelength of the microwave radiation ranges from about 915 MHz to about 2,450 MHz, and a microwave power of each microwave generator ranges from about 50 kW to about 150 kW. In some embodiments, said sterilizing occurs after a drying step.

In some embodiments, a protein feed product produced by processing the animal-based materials meets one or more regulatory standards. In some embodiments, the protein feed product is classifiable as a feed as defined in the “Code of Practice on Good Animal Feeding” (“Code”) of the Food and Agriculture Organization (FAO) of the United Nations, the entirety of which is hereby incorporated by reference for all purposes. The “Code” defines a feed as any single or multiple materials, whether processed, semi-processed or raw, which is intended to be fed directly to food producing animals.

As used herein, a conveyor or conveyor unit can be any vessel or mechanism that moves material from an inlet to an outlet. The material being heated can be carried in various examples by another type of conveyance mechanism, such as by an auger or various types of conveyor belts. Therefore, in some alternative embodiments a conveyorized modular industrial microwave power system can be employed instead of an auger-based system such as system 100. A conveyor unit can also be referred to more generally as an auger herein.

Based on power requirements, two or more microwave power modules or heating units can be installed on the same conveyor. To assure uniform heat distribution in a large variety of load configurations, a multimode cavity can be provided with a waveguide splitter with dual microwave feed points and mode stirrers.

In embodiments that use a conveyor belt, a belt material and configuration are selected based on the nature of the material or product being heated. Each end of the conveyor is preferably also provided with a special vestibule to suppress any microwave leakage. Air intake and exhaust vents or ports are provided for circulating air to be used in cases where vapors or fumes are developed during the heating process.

Unlike home microwave ovens, example industrial microwave-based heating systems contemplated herein preferably separate microwave generation from a heating/drying cavity such as a trough or housing. An example industrial microwave heating system can be constructed to use one or more microwave generator units. Example microwave generator and heating units come in 75 kW and 100 kW (output power) models. Using special ducts called waveguides or microwave guides, the microwave energy is carried to one or more industrial microwave cavities. In a conveyor belt-based embodiment, a conveyor belt, auger, etc. carries the material through the cavities. A simple example system may include one microwave generator and one cavity, while a larger and/or more complex system may have a dozen generators and six cavities. This inherent modularity provides great flexibility in scaling a system, or building systems, which can be easily expanded in the future.

These and other advantages will be apparent to those of ordinary skill in the art. While the various embodiments of the invention have been described, the invention is not so limited. Also, the method and apparatus of the present invention is not necessarily limited to any particular field, but can be applied to any field where an interface between a user and a computing device is applicable.

The disclosures of published PCT patent applications, PCT/US2017/023840 (WO2017165664), PCT/US2013/039687 (WO2013166489), PCT/US2013/039696 (WO2013166490), and PCT/US2020/040464 (WO2021003250), PCT/US2021/033145 (WO2022245348), and PCT/US2021/034241 (WO2022250663), and pending PCT applications: PCT/US2022/42334 (filed Sep. 1, 2022), and PCT/US2022/36331 (filed Jul. 7, 2022) are hereby incorporated by reference for all purposes.

In alternative embodiments, example microwave suppression flap(s) can be rigid and non-flexible, but can be attached to top portion using hinges or any other articulating hardware as known in the art. Alternative hardware and flap fastening arrangements are also contemplated.

Optionally, microwave heating disclosed herein can be continuous and/or pulsed or varied according to various material characteristics.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods, and materials are described below. All publications, patent applications, patents, and other references mentioned herein are incorporated by reference in their entirety to the extent allowed by applicable law and regulations. In case of conflict, the present specification, including definitions, will control.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention. Those of ordinary skill in the art that have the disclosure before them will be able to make modifications and variations therein without departing from the scope of the invention.

Selected embodiments of the present disclosure:

Embodiment 1. A system for processing animal-based material, comprising:
at least one microwave generator;
at least one microwave guide operatively connecting the at least one microwave generator to at least a first conveyor unit;
the first conveyor unit provided in a first housing that comprises at least one opening configured to receive microwave energy via a first microwave guide; and
wherein the first conveyor unit is configured to receive and process a quantity of animal-based material, which includes heating the animal-based material to a first temperature by applying microwave energy to the animal-based material within the first housing.
Embodiment 2. The system of embodiment 1, wherein the animal-based material comprises animal waste material.
Embodiment 3. The system of embodiment 2, wherein the animal waste material comprises manure.
Embodiment 4. The system of embodiment 2, wherein the animal waste material contains at least some solids.
Embodiment 5. The system of embodiment 1, wherein the animal-based material comprises animal meat or organs.
Embodiment 6. The system of embodiment 1, wherein the animal-based material after processing and heating is a product suitable for reuse, resale, and/or consumption.
Embodiment 7. The system of embodiment 1, wherein the heating the animal-based material at least partially converts the animal-based material to an animal bedding product.
Embodiment 8. The system of embodiment 1, wherein the heating the animal-based material at least partially converts the animal-based material to a fuel product.
Embodiment 9. The system of embodiment 1, wherein the heating the animal-based material at least partially converts the animal-based material to a fertilizer or land application product.
Embodiment 10. The system of any preceding embodiment, further comprising a second conveyor unit, the second conveyor unit provided in a second housing that comprises at least one opening configured to receive microwave energy via a second microwave guide, wherein the second conveyor is configured to receive and process the animal-based material, which includes heating the animal-based material to a second temperature greater than the first temperature by applying microwave energy to the animal-based material within the second housing.
Embodiment 11. The system of any preceding embodiment, wherein the at least one microwave generator comprises a plurality of microwave generators.
Embodiment 12. The system of any preceding embodiment, wherein the at least one microwave guide comprises a plurality of microwave guides.
Embodiment 13. The system of any preceding embodiment, wherein one or more additives are added to the animal-based material before, during, or after processing.
Embodiment 14. The system of any preceding embodiment, wherein the animal-based material being processed has an initial maximum particle size, and wherein the initial particle size is reduced to a second particle size by at least one of the first and second conveyors.
Embodiment 15. The system of any preceding embodiment, further comprising a third conveyor unit provided in a third housing that comprises at least one opening configured to receive microwave energy via a third microwave guide, and wherein the third conveyor is configured to receive and process the animal-based material, which includes heating the animal-based material to a third temperature greater than the second temperature by applying microwave energy to the animal-based material within the third housing.
Embodiment 16. The system of any preceding embodiment, further comprising a first loader unit configured to receive and feed the animal-based material to the first conveyor unit.
Embodiment 17. The system of any preceding embodiment, further comprising at least one microwave suppression system, comprising:
at least an inlet and an outlet; and
a tunnel within at least one of the inlet and outlet that comprises at least one flexible and/or movable microwave reflecting component within the tunnel, and
wherein at least a portion of the at least one movable microwave reflecting component is configured to be deflected as the animal-based material passes through the tunnel and then returning to a resting, closed position when the animal-based material is no longer passing through the tunnel.
Embodiment 18. The system of any preceding embodiment, wherein the movable microwave reflecting component is a mesh flap.
Embodiment 19. The system of any preceding embodiment, wherein the movable microwave reflecting component comprises stainless steel or aluminum.
Embodiment 20. The system of any preceding embodiment, wherein the movable microwave reflecting component is coated with a protective material.
Embodiment 21. The system of any preceding embodiment, wherein the protective material is selected from the group consisting of silicone, Teflon, polyurethane, and plastic.
Embodiment 22. The system of any preceding embodiment, wherein the movable microwave reflecting component comprises a plurality of strips.
Embodiment 23. The system of any preceding embodiment, wherein the movable microwave reflecting component comprises a plurality of chains.
Embodiment 24. The system of any preceding embodiment, further comprising at least a second microwave suppression system.
Embodiment 25. The system of any preceding embodiment, wherein at least one of the first, second, and third conveyor units comprises at least one helical auger.
Embodiment 26. The system of any preceding embodiment, further comprising a motor configured to rotate the at least one helical auger.
Embodiment 27. The system of any preceding embodiment, wherein the motor has a power rating of approximately 50-150 kilowatts.
Embodiment 28. The system of any preceding embodiment, wherein the motor has a power rating of approximately 70-130 kilowatts.
Embodiment 29. The system of any preceding embodiment, wherein the motor has a power rating of approximately 80-110 kilowatts.
Embodiment 30. The system of any preceding embodiment, wherein the motor has a power rating of approximately 90-100 kilowatts.
Embodiment 31. The system of any preceding embodiment, further comprising a mixer configured to receive the animal-based material being processed from a conveyor unit, wherein the animal-based material enters a different conveyor unit after exiting the mixer.
Embodiment 32. The system of any preceding embodiment, wherein the mixer is a pugmill, a drum mixer, or a mixing chamber.
Embodiment 33. The system of any preceding embodiment, further comprising a lifting conveyor configured to receive animal-based material being processed from the mixer and configured to lift the animal-based material vertically before the animal-based material enters a different conveyor unit.
Embodiment 34. The system of any preceding embodiment, wherein the animal-based material being processed comprises at least some drying of the animal-based material.
Embodiment 35. The system of any preceding embodiment, wherein after processing, the animal-based material is output as a food product.
Embodiment 36. The system of any preceding embodiment, wherein the food product is intended for consumption by humans.
Embodiment 37. The system of any preceding embodiment, wherein the food product is intended for consumption by animals.
Embodiment 38. The system of any preceding embodiment, wherein the animal-based material being processed contains at least some water.
Embodiment 39. The system of any preceding embodiment, wherein the animal-based material being processed contains ninety percent or less water by weight.
Embodiment 40. The system of any preceding embodiment, wherein the animal-based material being processed contains at least five percent water by weight.
Embodiment 41. The system of any preceding embodiment, wherein the animal-based material being processed contains at least ten percent water by weight.
Embodiment 42. The system of any preceding embodiment, wherein the animal-based material being processed contains between twenty and ninety percent water by weight.
Embodiment 43. The system of any preceding embodiment, wherein the animal-based material being processed contains between fifty and ninety percent water by weight.
Embodiment 44. The system of any preceding embodiment, further comprising at least one heat exchanger apparatus configured to recover a heat byproduct from the animal-based material being processed.
Embodiment 45. The system of any preceding embodiment, wherein the heat byproduct is recovered from the heating of the water within the animal-based material being processed.
Embodiment 46. The system of any preceding embodiment, wherein each conveyor unit is configured to receive between 1 and 30 microwave guides via corresponding openings.
Embodiment 47. The system of any preceding embodiment, wherein each conveyor unit is configured to receive between 7 and 10 microwave guides via corresponding openings.
Embodiment 48. The system of any preceding embodiment, wherein the animal-based material being processed receives about 0.33 and 0.44 kilowatts of microwave power per pound, including any moisture present within the animal-based material.
Embodiment 49. The system of any preceding embodiment, wherein the animal-based material being processed receives less than 0.33 kilowatts of microwave power per pound, including any moisture present within the animal-based material.
Embodiment 50. The system of any preceding embodiment, wherein each conveyor unit has a weight capacity of at least 500 pounds of animal-based material.
Embodiment 51. The system of any preceding embodiment, wherein each conveyor unit has a weight capacity of at least 8,500 pounds of animal-based material.
Embodiment 52. The system of any preceding embodiment, wherein each conveyor unit has a weight capacity of at least 40,000 pounds of animal-based material.
Embodiment 53. The system of any preceding embodiment, wherein the first conveyor unit comprises a baffle configured to restrict and shape the animal-based material being processed as it proceeds through the first housing.
Embodiment 54. The system of any preceding embodiment, wherein an additional material or composition is added to the animal-based material being processed.
Embodiment 55. The system of any preceding embodiment, wherein the animal-based material being processed has a maximum largest dimension of eight inches.
Embodiment 56. The system of any preceding embodiment, wherein the animal-based material being processed has a maximum largest dimension of six inches.
Embodiment 57. The system of any preceding embodiment, further comprising an impactor, shredder, mixer, mesh, brush, or other mechanical device configured to reduce a maximum largest dimension of the animal-based material being processed.
Embodiment 58. The system of any preceding embodiment, wherein the system processes between about 10 tons and about 1000 tons of animal-based material per hour.
Embodiment 59. The system of any preceding embodiment, wherein the system processes between about 50 tons and about 100 tons of animal-based material per hour.
Embodiment 60. The system of any preceding embodiment, wherein at least some of the animal-based material being processed is crushed or reduced in size within or prior to entering the first conveyor unit.
Embodiment 61. The system of any preceding embodiment, wherein the system is modular and portable.
Embodiment 62. The system of any preceding embodiment, wherein the system is contained within one or more trailers.
Embodiment 63. The system of any preceding embodiment, wherein the one or more trailers are transported to various processing locations on demand.
Embodiment 64. The system of any preceding embodiment, wherein at least one conveyor unit comprises a heated auger.
Embodiment 65. The system of any preceding embodiment, wherein the heated auger is a jacketed auger.
Embodiment 66. The system of any preceding embodiment, wherein at least one conveyor unit comprises a non-stick coating.
Embodiment 67. The system of any preceding embodiment, wherein at least one conveyor unit is thermally insulated.
Embodiment 68. The system of any preceding embodiment, wherein the animal-based material is heated to a temperature and duration such that the animal-based material is pasteurized.
Embodiment 69. A method of processing animal-based material, comprising:
receiving a quantity of animal-based material at a first conveyor unit provided in a first housing; and
performing a first processing step to the quantity of animal-based material within the first conveyor unit using at least one microwave generator coupled to the housing of the first conveyor unit, wherein the animal-based material is heated within the first conveyor unit.
Embodiment 70. The method of embodiment 69, further comprising:
receiving the quantity of animal-based material at a mixer, wherein a mixing step is performed to the animal-based material within the mixer.
Embodiment 71. The method of any preceding embodiment, wherein at least some of the animal-based material is mechanically processed, or otherwise reduced in size before or during the first processing step.
Embodiment 72. The method of any preceding embodiment, further comprising:
receiving the quantity of animal-based material at a second conveyor unit provided in a second housing; and
performing a second processing step to the quantity of animal-based material within the second conveyor unit using the at least one microwave generator coupled to the housing of the second conveyor, wherein the animal-based material is heated to a greater temperature in the second processing step than in the first processing step.
Embodiment 73. The method of any preceding embodiment, further comprising:
receiving the quantity of animal-based material at a third conveyor unit provided in a third housing; and
performing a third processing step to the quantity of animal-based material within the third conveyor unit using the at least one microwave generator coupled to the housing of the third conveyor, wherein the animal-based material is heated to a greater temperature in the third processing step than in the first or second processing steps.
Embodiment 74. The method of any preceding embodiment, wherein the quantity of animal-based material received at the mixer is received from a conveyor unit, and wherein the animal-based material enters a different conveyor unit after exiting the mixer.
Embodiment 75. The method of any preceding embodiment, wherein the at least first conveyor unit comprises a number and arrangement of conveyor units selected such that a desired result is reached.
Embodiment 76. The method of any preceding embodiment, wherein at least two conveyor units are arranged in series.
Embodiment 77. The method of any preceding embodiment, wherein at least two conveyor units are arranged in parallel.
Embodiment 78. The method of any preceding embodiment, wherein a processing speed of the at least one conveyor unit is adjusted based on the series or parallel arrangement.
Embodiment 79. The method of any preceding embodiment, wherein the processing speed can be reduced to increase heating, or can be increased to reduce heating of the animal-based material being processed in the at least one conveyor unit.
Embodiment 80. The method of any preceding embodiment, wherein for a given processing speed, two or more conveyor units operating in parallel increases an animal-based material throughput based at least on the number of parallel conveyor units.
Embodiment 81. The method of any preceding embodiment, further comprising using a microwave radar of a frequency different than any heating microwaves to perform at least a level measurement.
Embodiment 82. The method of any preceding embodiment, wherein based on the level measurement at least one of a processing speed and heating power is adjusted.
Embodiment 83. A product made by any system or method of any preceding embodiment.
Embodiment 84. A product, apparatus, method, or system of any preceding embodiment wherein processing of animal-based material is continuous.
Embodiment 85. A product, apparatus, method, or system of any preceding embodiment wherein processing of animal-based material is in batches.
Embodiment 86. A method for portably providing animal-based material processing upon demand, comprising:
receiving a request for processing a first quantity of animal-based material at a first location;
determining that the first location has a first group of characteristics that include at least a distance from the first location to an external power source of a first power output;
deploying a portable system for processing animal-based material at the first location based on at least the first quantity of animal-based material and the first group of characteristics, the portable system comprising:
at least one power generator configured to provide at least the first power output,
at least one microwave generator operatively coupled to the power generator,
at least one conveyor unit configured to receive and process a quantity of animal-based material to achieve at least a target temperature for a target time; and
   applying microwave energy to the animal-based material within the conveyor unit of the portable system.
   Embodiment 87. The method of embodiment 86, wherein the processing of the animal-based material operates continuously.
   Embodiment 88. The method of embodiment 86, wherein the processing of the animal-based material operates in batches.
   Embodiment 89. A microwave suppression system, comprising:
   at least an inlet and an outlet; and
   a tunnel within at least one of the inlet and outlet that comprises at least one movable mesh flap within the tunnel,
   wherein the at least one movable mesh flap is configured to absorb, deflect, or block microwave energy, and
   wherein the at least one movable mesh flap is configured to be deflected as an animal-based material passes through the tunnel and then to return to a resting, closed position when the animal-based material is no longer passing through the tunnel.
   Embodiment 90. The microwave suppression system of embodiment 89, wherein the movable mesh flap comprises stainless steel.
   Embodiment 91. The microwave suppression system of embodiment 89, wherein the microwave suppression system operates to treat animal-based material continuously.
   Embodiment 92. An apparatus for treating animal-based material, comprising:
   a conveyor unit comprising a helical auger having an auger shaft provided along an auger rotational axis, the auger configured to rotate in a direction such that a quantity of animal-based material received at the conveyor unit is caused to be transported according the auger rotational axis; and
   at least one microwave energy generator, each microwave energy generator being operatively connected to a respective microwave guide configured to cause microwaves emitted by the microwave energy generator to heat the animal-based material within the conveyor unit by converting the microwaves to heat when absorbed by at least a portion of the quantity of animal-based material within the conveyor unit;
   wherein the quantity of animal-based material is heated using the microwave energy, and wherein the quantity of animal-based material is caused to exit the conveyor unit after being heated according to a target specification.
   Embodiment 93. The apparatus of embodiment 92, wherein the apparatus treats the animal-based material continuously.
   Embodiment 94. The apparatus of embodiment 92, wherein the auger shaft defines an internal auger fluid path provided along the auger rotational axis, and further comprising a fluid management device configured to heat the auger and transfer heat to the quantity of animal-based material through the auger, wherein the quantity of animal-based material is heated using a combination of the microwave energy and fluidic heat.
   Embodiment 95. The apparatus of embodiment 92, further comprising:
   a material inlet and a material outlet;
   a tunnel within at least one of the material inlet and material outlet that comprises a microwave suppression system;
   at least one movable mesh flap within the tunnel, wherein the at least one mesh flap is configured to absorb, deflect, or block microwave energy, and wherein the at least one movable mesh flap is configured by be deflected as the animal-based material passes through the tunnel and then returning to a resting, closed position when the animal-based material is no longer passing through the tunnel.
   Embodiment 96. The apparatus of embodiment 95, wherein the movable mesh flap comprises stainless steel.
   Embodiment 97. A method of treating animal-based material using microwave energy, comprising:
   receiving a quantity of animal-based material at a conveyor unit comprising an auger, wherein the animal-based material passes through at an inlet microwave suppression tunnel before entering the conveyor unit;
   transporting the quantity of animal-based material along the conveyor unit by causing the auger to rotate;
   heating the quantity of animal-based material within the conveyor unit using at least one microwave generator operatively connected to a respective microwave guide configured to cause microwaves emitted by the microwave energy generator to heat the quantity of animal-based material within the conveyor unit by converting the microwaves to heat when absorbed by at least a portion of the quantity of animal-based material within the conveyor unit; and
   causing the heated quantity of animal-based material to exit the conveyor unit through an outlet microwave suppression tunnel, wherein the quantity of animal-based material that exits the conveyor unit is a usable animal product or precursor to a usable animal product.
   Embodiment 98. The method of embodiment 97, wherein the quantity of animal-based material is heated to a target temperature before being caused to exit the conveyor unit.
   Embodiment 99. The method of embodiment 97, wherein the quantity of animal-based material is heated such that it is sterile and is substantially free of pathogens and microbes.
   Embodiment 100. The method of embodiment 97, wherein the inlet suppression tunnel comprises:
   at least one inlet movable mesh flap within the inlet suppression tunnel,
   wherein the at least one inlet movable mesh flap is configured to absorb, deflect, or block microwave energy, and
   wherein the at least one inlet movable mesh flap is configured to be deflected as the quantity of animal-based material passes through the inlet suppression tunnel and then to return to a resting, closed position when the quantity of animal-based material is no longer passing through the inlet suppression tunnel.
   Embodiment 101. The method of embodiment 100, wherein the inlet movable mesh flap comprises stainless steel.
   Embodiment 102. The method of embodiment 97, wherein the outlet suppression tunnel comprises:
   at least one outlet movable mesh flap within the outlet suppression tunnel, wherein the at least one outlet movable mesh flap is configured to absorb, deflect, or block microwave energy, and
   wherein the at least one outlet movable mesh flap is configured to be deflected as the quantity of animal-based material passes through the outlet suppression tunnel and then to return to a resting, closed position when the quantity of animal-based material is no longer passing through the outlet suppression tunnel.
   Embodiment 103. The method of embodiment 102, wherein the outlet movable mesh flap comprises stainless steel.
   Embodiment 104. The method of embodiment 97, wherein the treating of the animal-based material operates continuously.
   Embodiment 105. A method for sharing portable animal-based material processing, comprising:
   receiving a request for processing a first quantity of animal-based material at a first location and a second location separate from the first location;
   determining that the first location has a first group of characteristics;
   determining that the second location has a second group of characteristics
   deploying a portable system for processing animal-based material at the first location or the second location based on at least the first quantity of animal-based material and the first group of characteristics or the second quantity of animal-based material and the second group of characteristics, the portable system comprising:
at least one power generator configured to provide at least the first power output,
at least one microwave generator operatively coupled to the power generator,
at least one conveyor unit configured to receive and process a quantity of animal-based material to achieve at least a target temperature for a target time; and applying microwave energy to the first or second quantity animal-based material within the conveyor unit of the portable system.
   Embodiment 106. The method of embodiment 105, wherein the first group of characteristics comprises first end result requirements and animal-based processing specifications of the first location, and the second group of characteristics comprises second end result requirements and animal-based processing specifications of the second location.
   Embodiment 107. A product, apparatus, method, or system of any preceding embodiment wherein the received animal-based material is flowable.
   Embodiment 108. A product, apparatus, method, or system of any preceding embodiment wherein the processing the animal-based material produces an output animal product that comprises solids.
   Embodiment 109. A product, apparatus, method, or system of any preceding embodiment wherein the processing the animal-based material produces an output animal product that comprises animal bedding.
   Embodiment 110. A product, apparatus, method, or system of any preceding embodiment wherein heating the animal-based material to a first temperature by applying microwave energy to the animal-based material causes at least a desired chemical reaction within the animal-based material.

1. A system for processing animal-based material, comprising:
a material inlet and a material outlet;
at least a first conveyor unit associated with at least one of the material inlet and the material outlet;
at least one microwave generator;
at least a first microwave guide operatively connecting the at least one microwave generator to at least the first conveyor unit,
wherein the first conveyor unit is provided in a first housing that comprises at least one microwave opening configured to receive microwave energy via at least the first microwave guide; and
at least one microwave suppression system associated with the first conveyor unit, each microwave suppression system comprising:
a tunnel associated with at least one of the material inlet and the material outlet, and
at least one flexible and/or movable microwave reflecting component comprised within the tunnel,
wherein at least a portion of the at least one microwave reflecting component is configured to be deflected as a quantity of animal-based material passes through the tunnel and then to return to a resting, closed position when the animal-based material is no longer passing through the tunnel,
wherein the first conveyor unit is configured to receive and process the animal-based material, the processing including heating the animal-based material to at least a first temperature by applying microwave energy to the animal-based material within the first housing.

2. The system of claim 1, wherein the animal-based material comprises animal waste material, wherein the animal waste material comprises at least one of animal manure, urine, hair, or any other waste material produced by animals.

3. The system of claim 1, wherein the animal-based material comprises animal body parts.

4. The system of claim 1, wherein the first temperature is a target temperature based on a target specification of the animal-based material after processing.

5. The system of claim 1, wherein the animal-based material is heated to the first temperature for a first time period within the first housing.

6. The system of claim 1, wherein the animal-based material is heated such that it is substantially sterile and is substantially free of microbial and pathogen matter.

7. The system of claim 1, wherein the animal-based material is heated to a temperature and duration such that the animal-based material is pasteurized.

8. The system of claim 1, wherein the animal-based material after processing and heating is a product suitable for reuse, resale, and/or consumption or usage by humans or animals.

9. The system of claim 1, wherein the heating the animal-based material at least partially converts the animal-based material to an animal bedding, fuel, compost, or fertilizer product.

10. The system of claim 1, wherein, after processing, the animal-based material is caused to exit the first conveyor unit as a usable animal-derived product or precursor to a usable animal-derived product.

11. The system of claim 1, wherein the system is configured to process the animal-based material continuously, and wherein a processing speed of the system is adjustable such that the speed can be reduced to increase heating, or can be increased to reduce heating of the animal-based material being processed within the first conveyor unit.

12. The system of claim 1, further comprising a second conveyor unit, the second conveyor unit provided in a second housing that comprises at least one microwave opening configured to receive microwave energy via at least a second microwave guide, wherein the second conveyor is configured to receive and process the animal-based material, which includes heating the animal-based material to a second temperature greater than the first temperature by applying microwave energy to the animal-based material within the second housing.

13. The system of claim 1, further comprising a mechanical processing apparatus associated with the first conveyor unit, wherein the animal-based material enters the first conveyor unit before entering or after exiting the mechanical processing apparatus, wherein the mechanical processing apparatus is a mill, a crusher, a mixer, a loader unit, an impactor, a shredder, a mesh, a screen, a brush, a sorting apparatus, a blender, a lifting apparatus, a homogenizing apparatus, or an apparatus configured to reduce a maximum largest dimension and/or increase the density of the animal-based material being processed.

14. The system of claim 1, wherein the movable microwave reflecting component is a mesh flap comprising stainless steel or aluminum.

15. The system of claim 1, further comprising at least a second microwave suppression system.

16. The system of claim 1, wherein the animal-based material to be processed contains at least a first water percentage by weight, and the first water percentage by weight of the animal-based material is reduced to a second water percentage by weight lower than the first water percentage by weight during or after the processing.

17. The system of claim 1, further comprising at least one heat exchanger apparatus configured to recover a heat byproduct from the animal-based material being processed.

18. The system of claim 1, wherein an additional material or composition is added to the animal-based material being processed.

19. An apparatus for processing animal-based material, comprising:
a material inlet and a material outlet;
a conveyor unit comprising an auger having an auger shaft provided along an auger rotational axis, the auger configured to rotate in a direction such that a quantity of animal-based material received at the conveyor unit is caused to be transported according to the auger rotational axis;
at least one microwave energy generator, each microwave energy generator being operatively connected to at least a respective microwave guide configured to cause microwaves emitted by the microwave energy generator to heat the animal-based material within the conveyor unit by converting the microwaves to heat when absorbed by at least a portion of the animal-based material within the conveyor unit; and
at least a first microwave suppression system comprising a tunnel associated with at least one of the material inlet and material outlet, wherein the first microwave suppression system comprises at least one flexible and/or movable microwave reflecting component within the tunnel, wherein the at least one microwave reflecting component is configured to absorb, deflect, or block microwave energy, and wherein the at least one microwave reflecting component is configured to be deflected as the animal-based material passes through the tunnel and then to return to a resting, closed position when the animal-based material is no longer passing through the tunnel,
wherein the animal-based material is heated using microwave energy, and wherein the animal-based material is caused to be heated to a target specification by the microwaves emitted by the at least one microwave generator.

20. A method of processing animal-based material using microwave energy, comprising:
receiving a quantity of animal-based material at a conveyor unit, wherein the animal-based material passes through an inlet microwave suppression tunnel before entering the conveyor unit, wherein the inlet microwave suppression tunnel comprises at least one flexible and/or movable inlet microwave reflecting component within the inlet microwave suppression tunnel, and wherein the at least one inlet microwave reflecting component is configured to absorb, deflect, or block microwave energy;
deflecting the at least one inlet microwave reflecting component as the animal-based material passes through the inlet microwave suppression tunnel and then optionally returning the at least one inlet microwave reflecting component to a resting, closing position when the animal-based material is no longer passing through the inlet microwave suppression tunnel;
transporting the animal-based material using at least the conveyor unit;
heating the animal-based material within at least the conveyor unit using at least one microwave generator operatively connected to a respective microwave guide configured to cause microwaves emitted by the microwave energy generator to heat the animal-based material within at least the conveyor unit by converting the microwaves to heat when absorbed by at least a portion of the animal-based material within at least the conveyor unit; and
causing the animal-based material to exit through an outlet microwave suppression tunnel after the animal-based material is heated such that the animal-based material: a) reaches a first temperature, b) undergoes a reaction, and/or c) reaches a target specification within at least the conveyor unit.
